(19) United States
(12) Patent Application Publication
(10) Pub. No.: US 2020/0323520 A1
(43) Pub. Date: Oct. 15, 2020

(54) SMALL TUBE TISSUE BIOPSY

(71) Applicant: GW Medical LLC, Pleasanton, CA (US)

(72) Inventors: E. Skott GREENHALGH, Gladwyne, PA (US); Michael P. WALLACE, Pleasanton, CA (US)

(21) Appl. No.: 16/847,647

(22) Filed: Apr. 13, 2020

Related U.S. Application Data
(60) Provisional application No. 62/832,784, filed on Apr. 11, 2019.
(60) Provisional application No. 62/929,761, filed on Nov. 1, 2019.

(51) Int. Cl. A61B 10/02

(52) U.S. Cl. CPC A61B 10/0291

(57) ABSTRACT

Apparatuses and methods for biopsying body vessels having small lumen, such as Fallopian tubes. In particular, described herein are apparatuses (e.g., systems and devices) and methods that evert a textile biopsying member to sample tissue from a vessel or conduit such as the fallopian tubes without applying substantial shear force.

### CROSS REFERENCE TO RELATED APPLICATIONS

This patent application claims priority to U.S. Provisional Patent Application No. 62/832,784, filed on Apr. 11, 2019, titled “SMALL TUBE TISSUE BIOPSY,” and U.S. Provisional Patent Application No. 62/929,761, filed on Nov. 1, 2019, titled “SMALL TUBE TISSUE BIOPSY.” Each of these application is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are apparatuses and methods for biopsying body vessels having small lumen, such as Fallopian tubes. In particular, described herein are apparatuses (e.g., systems and devices) and methods that evert a textile biopsying member to sample tissue from a vessel or conduit such as the fallopian tubes without applying substantial shear force.

### BACKGROUND

A biopsy is the removal of tissue in order to examine it for disease. Usually, a biopsy is performed to examine tissue for disease. Biopsies are frequently used to diagnose cancer, but they can help identify other conditions such as infections and inflammatory and autoimmune disorders. They may also be done to match organ tissue before a transplant and to look for signs of organ rejection following a transplant. The tissue samples can be taken from any part of the body, including small channels, ducts and tubes. Biopsies are performed in many areas of the body and for many reasons. In particular, biopsies may be taken from any duct, channel, or vessel within the body, such as (but not limited to) the fallopian tubes (e.g., uterine tubes).

For example, an endometrial biopsy may be used when looking for the cause of abnormal uterine bleeding, to examine the lining of the uterus and to diagnose cancer. This type of biopsy can be performed by using a small needle-like device to capture a sample or by using a tool to scrape some of the lining for examination. Ovarian cancer is a significant disease in women. Early detection of ovarian cancer may be difficult due to a lack of effective screening tests, such that ovarian cancer may not be diagnosed until the disease has reached advanced stages, limiting treatment options. Screening for ovarian cancer may typically include a surgical procedure for obtaining cell samples for diagnosis. For example, because the ovaries are intra-abdominal, laparoscopic or open surgery (laparotomy) may be performed to access the ovaries. Any surgical procedure increases a risk to the patient, including but not limited to experiencing an adverse reaction, and/or requiring significant recovery time. Additionally, an ovary biopsy may expose the patient to additional risk of potentially spreading diseased (e.g., cancerous) cells.

Thus, there exists a need for devices and processes to allow samples to be obtained from body ducts and tubes, such as the fallopian tubes, for evaluation of tissue, including but not limited to the detection of ovarian cancer, in a less invasive and controlled fashion and, particularly without the need for a skin incision.

### SUMMARY OF THE DISCLOSURE

Described herein biopsy apparatuses configured to taking biopsy (e.g., tissue, cell, etc.) samples from within a small-diameter lumen of a body. These biopsy apparatuses include devices, which may be referred to herein as small-tube biopsy devices, and system including them. These apparatuses may gently remove a biopsy sample from the wall(s) of a vessel lumen without risk of rupture of the vessel and with minimal irritation of the vessel.

In general, these apparatuses may include an inner tubular member that is concentrically and movably held within an outer tubular member. A biopsy collector can be deployed from the distal end of the apparatus and may be expandable so that, when the inner member is moved relative to the outer member by a control on a proximal handle, the biopsy collector expands outward into and against the walls of the lumen.

For example, described herein are small-tube biopsy devices having: an inner member coaxially arranged within an outer member, so that the inner member may move axially in a distal and proximal direction relative to the outer member; a biopsy collector comprising a knitted or woven textile configured to collect biopsy tissue, wherein the biopsy collector is connected at a first end to the inner member and at a second end to the outer member; and a proximal handle coupled to the inner member and the outer member and configured to move the inner member axially relative to the outer member to expand the biopsy collector.

The knitted or woven textile may generally be configured for biopsy collection. For example, the knitted or woven textile may have a roughness sufficient for removing tissue, and/or a porosity that is configured to enhance collection of biopsy tissue.

In addition, in any of these variations described herein, the device may initially be configured so that knitted or woven textile forming the biopsy collector (e.g., the biopsy collector) is pre-loaded for deployment within the inside of outer member; all or the majority of it (e.g., greater than 75%, greater than 80%, greater than 85%, greater than 90%, greater than 95%, etc.) is initially pre-loaded within the outer member, with the inner member retracted proximally. The knitted or woven textile may configured to have a sufficient column strength to allow it to be pushed distally out of the outer member (which may be a flexible tube, such as a cannula, or coil) so that it may evert as it passes beyond the distal end of the outer member. The knitted or woven textile may be configured to prevent it from rolling against the distal end of the outer member, which may otherwise contaminate the biopsy collector and/or may dislodge any biopsy material when the knitted or woven textile is withdrawn back into the outer member. The knitted or woven material may be configured to prevent it from rolling against the distal end of the outer member in one or more of a variety of ways, including by attaching one end or end region of the knitted or woven material to an inner lumen wall of the outer member (including attaching it in an inverted configuration relative to the attachment of the other end or end region of the knitted or woven material that is attached to the inner member. Alternatively or additionally, the knitted or woven material may be biased to roll and invert when driven distally. Alternatively or additionally, the knitted or woven material may be configured to be formed having have a specified range of filament shapes, sizes, count, weave angle, etc., as described herein. Filaments shapes can be round or flat, the size of the filaments are and number of filaments may be related to the internal diameter of the delivery system (e.g., the outer member). A smaller diameter delivery system, such as an outer member configured as a catheter or coil having an inner diameter (ID) of 1.5 mm or less may be used with an everting biopsy collector (e.g., which may preferably be a textile structure) formed from smaller filaments, such as wire filament or combined wire/polymer filaments, such as filaments between 0.001″ to 0.05″. The wire filaments may be formed, e.g., of Nitinol, spring steel and it's alloys, etc. In addition, the number of filaments per cross sectional area may be low.

In general the stability of the everted biopsy collector (e.g., textile structure) may benefit from higher column force resistance. Higher column force may provide a stable structure to aid in pushing the textile while everting. Thus, the biopsy collector may be configured to be a formed of a “stacked” high density knit, high density weave, and the like, typically having a low braid angle.

As mentioned, the knitted or woven textile may be configured to collect and/or retain the biopsy tissue. For example, the knitted or woven textile may have pores that are within a range of, e.g., between about 0.002″ and 0.05″. Variations of the biopsy collector that provide a higher radial force may expand the textile structure outward into the vessel wall increasing collection efficiency. As described in greater detail below, this may also allow these apparatuses to open a closed-off biological lumen/vessel and/or help maintain patency of the lumen/vessel. Touching wall and creating a shear force at the desired time (e.g., after deployment, but preferably not before) may also greatly improve sampling. Increasing radial force may be accomplished by one or more of: forming the biopsy collector from using large wire diameters/cross section, using more wires (e.g., greater knit, weave, braid per given diameter), the materials used for the wires (e.g., Nitinol, Steel, Elgiloy, MP35n, etc., generally stiffer may be preferred), and finally the annealed shape of the final everted biopsy collector member. The apparatuses described herein may be configured so that they are annealed to have an oversized outer diameter compared to the vessel in which they are deployed, such as, for example, an inner diameter that is between 25% to 100% bigger in the annealed shape than the target vessel. For example the annealed shape may be between 1 mm and 2 cm (e.g., between 2 mm and 1.5 cm, between 2.5 mm and 1.25 cm, etc.). Any of the apparatuses described herein may have a large pore size. For example, the pore size may be between about 0.002″ and 0.05″ for (e.g., between 0.005″ and 0.05″, between about 0.0075″ and 0.05″, between about 0.01″ and 0.05″, between about 0.02″ and 0.05″, between about 0.03″ and 0.05″, between about 0.005″ and 0.04″, between about 0.005″ and 0.03″, etc.). In general, any of the methods and apparatuses described herein may include a biopsy collector that comprises a co-knit, co-weave, and/or co-braid of Nitinol and one or more fluffy polymer yarn. For example, the hybrid structure (co-knit, co-weave, or co-braid) may be a mix of a radial force creating element (such as a Nitinol wire, for example) and one or more tissue grabbing/storing element such as a polymer (e.g., polyester).

For example, a small-tube biopsy device may include: an inner member coaxially arranged within an outer member, so that the inner member may move axially in a distal and proximal direction relative to the outer member; a biopsy collector comprising a knitted or woven textile configured to collect biopsy tissue, wherein the biopsy collector is connected at a first end to the inner member and at a second end to the outer member and wherein a majority of the biopsy collector is retained within a lumen of the outer member in an un-deployed configuration; and a proximal handle coupled to the inner member and the outer member and configured to move the inner member distally relative to the outer member to drive the biopsy collector out of a distal end of the outer member so that it everts beyond the distal end of the outer member without rolling against it and expands radially into a deployed configuration.

In some variations the second end of the biopsy collector is not attached to the outer member. For example, a small-tube biopsy device may include: an inner member coaxially arranged within an outer member, so that the inner member may move axially in a distal and proximal direction relative to the outer member; a biopsy collector comprising a knitted or woven textile configured to collect biopsy tissue, wherein the biopsy collector is connected at a first end to the inner member and wherein a second end is biased to expand radially and invert and roll over itself (and in particular, invert over itself without rolling against a distal end of the outer member) as it is driven distally out of the outer member, further wherein the majority (e.g., 75% or more, 80% or more, 90% or more, etc.) of the knitted or woven textile is contained within the outer member in a pre-deployed configuration; and a proximal handle coupled to the inner member and the outer member and configured to move the inner member axially relative to the outer member to expand the biopsy collector.

As mentioned, any of the biopsy collectors may include a knitted or woven textile; in some variations the knitted or woven textile is attached to another material, such as metal or other material that may support the knitted or woven textile. For example, the knitted or woven textile may be attached as a sleeve, cover, layer, etc., connected to an additional expandable frame.

The knitted or woven textile may be generally formed of any appropriate material, including polymeric material, metals (e.g., shape memory materials), fibrous materials (natural or artificial fibrous materials, including cottons, collagens, etc.). The material may be configured, including by treatment, coating, impregnation, etc., with a material that enhances the ability of the knitted or woven textile to capture (and in some cases remove) biopsy material, such as cells and/or tissues.

In any of the apparatuses described herein, the apparatus is configured for use with a guidewire. Thus, the inner member and the biopsy collector may be formed to include a continuous longitudinal passage for a guidewire or guide catheter. Thus, any of these devices may include a guidewire lumen thought the inner member and the biopsy collector.

As mentioned above, in some variations, the biopsy collector is connected at the first end to a distal end (or end region) of the inner member. The biopsy collector may be connected at the second end to a distal end (or end region) of the inner member. The connection may be a rigid connection, in which the two parts are affixed together, or it may be a movable coupling, in which the biopsy connector is constrained for axial movement, but allowed to rotate (e.g., relative to the inner member when connected to the inner member or relative to the outer member for the connection to the outer member).

In general the proximal handle may be configured to move the inner member axially relative to the outer member to expand the biopsy collector so that the outer diameter of the biopsy collector is enlarged to contact the inner walls of a body lumen. For example, the handle may include a control (slider, knob, etc.) that drives the inner member movement relative to the outer member for deploying from the un-deployed configuration in which the biopsy collector (including the knitted or woven textile material) is held within the inner lumen of the outer member, including e.g., against the inner wall of the outer member. For example, the proximal handle may comprise a control configured to move the inner member axially distally and proximally relative to the outer member while holding the outer member in a fixed position. Any appropriate control may be used, including a mechanical, electrical or both (e.g., a slider, a knob, a lever, and a trigger).

In some variations, as mentioned above, the biopsy collector (e.g. the knitted or woven textile) may be attached to an inner surface of the outer member. The biopsy collector may be attached to an inner surface (e.g., the inner luminal surface) of the outer member in an inverted orientation compared to the attachment of the biopsy collector to the inner member.

Also described herein are methods of collecting biopsy material from within a body lumen using any of the devices described herein. For example, a method of collecting biopsy material (cells, tissue, etc.) from a wall of a body lumen may include: advancing a biopsy device distally towards the body lumen; moving an inner member of the biopsy device distally within an outer member of the biopsy device so that a knitted or woven textile that is attached to a distal region of the inner member and to a distal region of the outer member rolls out of a distal end of the outer member and expands against an inner wall of the body lumen; moving the knitted or woven textile relative to the inner wall of the body lumen to capture cellular material from the body lumen on the knitted or woven textile; and withdrawing the inner member of the biopsy device proximally so that the knitted or woven textile is drawn back into the lumen of the outer member.

The body lumen may be any appropriate lumen, such as, e.g., a fallopian tube, a urethra, etc.

In any of these methods, advancing the biopsy device may comprises passing the biopsy device over a guidewire positioned in the body lumen. Thus, the guidewire (or guide catheter) may be positioned first, and the biopsy device placed over the guidewire (or guide catheter).

In general, any of these methods may include inverting the biopsy collector (e.g., the knitted or woven textile) as it rolls out of the distal end of the outer member, typically after passing beyond the distal end opening of the outer member. As mentioned above, it may invert to form a torus-like structure (against the walls of the small-diameter body lumen). For example, the knitted or woven textile forms a torus as it rolls out of the distal end of the outer member.

The biopsy may be taken by moving the biopsy collector (e.g., the knitted or woven textile) relative to the inner wall of the body lumen, such as, but not limited to a fallopian tube. This may pull the exposed surface of the biopsy collector against the lumen wall(s), capturing biopsy material. In some variations, this may be accomplished by securing the inner member and the outer member together and drawing the biopsy device proximally. Alternatively or additionally, the biopsy collector may be rotated relative to the body lumen to move against the wall of the lumen and collect biopsy material; this may be accomplished in some variations by rotating the inner member and/or rotating the entire device. Thus moving the knitted or woven textile relative to the inner wall of the body lumen comprises one or more of: rotating the biopsy collector (e.g., at least the knitted or woven textile) relative to the inner wall of the body lumen, pulling the biopsy collector (e.g., knitted or woven textile) proximally relative to the inner wall of the body lumen, and/or pushing the biopsy collector (e.g., knitted or woven textile) distally relative to the inner wall of the body lumen.

Once the biopsy sample has been collected, e.g., onto the knitted or woven material of the biopsy collector, the biopsy collector maybe retracted back into the outer member by withdrawing the inner member proximally to roll the biopsy collector back into the lumen of the outer member. The outer member and retracted biopsy collector may then be withdrawn. Alternatively or additionally, the biopsy collector may be housed within a delivery catheter for removal from the body to prevent contamination. For example, in some variations the inner member may be retracted proximally to withdraw the biopsy collector into the outer member completely or partially, and the outer member and inner member, and the biopsy collector may be retracted into a protective sheath, catheter, etc. (e.g., a delivery catheter); this same protective sheath, catheter, etc. may be used for deploying the device into body.

Also described herein are variations of the small-tube biopsy devices in which the biopsy collector is a coil. For example, described herein are small-tube biopsy device comprising: an inner member coaxially arranged within an outer member, so that the inner member may move relative to the outer member; a biopsy collector configured to collect biopsy tissue and comprising a coil helically wound around the inner member, wherein the biopsy collector is connected at a first end to the inner member and at a second end to the outer member; and a proximal handle coupled to the inner member and the outer member and configured to rotate the inner member relative to the outer member to expand the biopsy collector.

In this example, the inner member may be rotated to open or close the helically wound spring forming at least part of the biopsy collector. The helically wound coil may be tightened (to form a smaller diameter) or loosened (to form a larger diameter). Thus, in some variations, both ends or end regions of the biopsy collector may be attached to the inner member (at a first end) and the outer member (at a second end).

As mentioned above, any of the apparatuses, including devices, described herein may include a liner (a sleeve, a layer, etc.) for collecting the biopsy material. For example, the methods described herein may include a liner or similar material coupling the coil and configured to collect biopsy tissue. In some variations the liner or sleeve is attached over the distal end of the biopsy collector. In some variations the liner is within the lumen formed by the biopsy collector coil. In some variations the line encloses all or a portion of the coil of the biopsy collector. As mentioned, the liner may be configured to capture and/or retain the biopsy material. For example, the liner may comprise a woven or knitted material.

Also described herein are methods of collecting biopsy material from within a body lumen using a device that includes an expandable coil For example, the methods may include: advancing a biopsy device distally into the body lumen; rotating an inner member of the biopsy device within an outer member of the biopsy device in a first direction so that a coil that is attached to a distal region of the inner member and attached to a distal region of the outer member expands against an inner wall of the body lumen, wherein the coil is helically wound around a distal portion of the inner member that is outside of outer member; moving the coil relative to the inner wall of the body lumen to capture cellular material from the body lumen; and rotating the inner member of the biopsy device in a second direction so that the coil is contracted.

As mentioned, moving the coil may comprise moving the coil so that a liner coupled to the coil moves against the inner wall and captures the cellular material.

In any of the methods of using a device having a coil described herein, the method may include withdrawing the coil proximally into a lumen of a second outer member to protect the collected tissue.

Advancing the biopsy device may comprise passing the biopsy device over a guidewire positioned in the body lumen.

Also described herein are apparatuses and methods of using them that are configured to be screwed into the lumen to take a biopsy. For example, described herein are apparatuses such as small-tube biopsy devices comprising: an inner member coaxially arranged within an outer member, so that the inner member may move relative to the outer member; a helical biopsy collector configured to collect biopsy tissue and comprising a coil helically wound around the inner member, wherein the biopsy collector is connected to the inner member; and a proximal handle coupled to the inner member and the outer member and configured to rotate the inner member relative to the outer member.

As mentioned, any of these devices may include a liner coupled with the coil and configured to collect biopsy tissue. The liner may be formed of a woven or knitted material.

A method of collecting biopsy material from within a body lumen using such a device may include, for example: advancing a biopsy device distally towards the body lumen; rotating and distally advancing an inner member of the biopsy device relative to an outer member of the biopsy device so that a coil that is attached to the inner member screws against an inner wall of the body lumen and advances within the body lumen, wherein the coil is helically wound around a distal portion of the inner member that is outside of outer member; capturing cellular material from the body lumen as the coil is rotated and distally advanced; and withdrawing the inner member proximally. Capturing the cellular material may comprises capturing the cellular material in a liner coupled to the coil.

Withdrawing the coil proximally may comprise withdrawing the inner member proximally into a lumen of a second outer member to protect the collected tissue. Advancing the biopsy device may comprises passing the biopsy device over a guidewire positioned in the body lumen.

For example, described herein are methods of collecting biopsy material (e.g., from within a body lumen, such as from within a fallopian tube), which may include: advancing a biopsy device distally towards an opening into the body lumen (e.g., fallopian tube); extending a tube of braided textile distally into the fallopian tube by moving an inner member of the biopsy device distally within an outer member of the biopsy device, wherein a first end region of the braided textile is attached to a distal region of the inner member and a second end region of the braided textile is attached to a distal region of the outer member, so that the tube of braided textile extends distally from a distal end of the outer member and everts over itself as it extends into the fallopian tube; capturing cellular material from the fallopian tube on pores within the braided textile; and withdrawing the inner member of the biopsy device proximally so that the tube of braided textile is inverted and drawn proximally back into the lumen of the outer member. Capturing the cellular material may include moving the tube of braided textile against an inner wall of the fallopian tube to capture the cellular material.

Advancing may include advancing the biopsy device with the tube of braided textile completely within the outer member.

Capturing cellular material from the body lumen may include applying compressive force on the braided textile to form a stable column of braided material in an un-inverted configuration within the outer member. The braided textile (e.g., tube of braided textile) may be configured to provide column strength to prevent buckling or bending, e.g., by having (e.g., when there are between 40-50 strands/threads forming the braided textile) a braid angle of 25 degrees or less on the braided textile in the un-deployed configuration within the outer member. The outer diameter of the braided tube in the un-deployed configuration (under compression) may also be greater than 90% the inner diameter of the outer member.

The tube of braided textile may form a distally-extending torus as it extends out of the distal end of the outer member. In some variations, capturing comprises holding the outer member relative fixed and advancing the inner member distally to extend the tube of braided textile distally into the fallopian tube. Capturing may also include moving the tube of braided textile by: rotating the tube of braided textile relative to the fallopian tube, pulling the tube of braided textile proximally relative to the fallopian tube, and/or pushing the braided textile distally relative to the fallopian tube.

Although the methods and apparatuses (including systems and devices) described herein are directed in particular for biopsying the fallopian tubes, any of these devices and methods may be adapted for sampling other region, and particular tubular, body regions, such as the mouth, nasal passages, ear canals, esophagus, brachial passages, rectum, small intestine, etc.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. lA and 1B show an example of a biopsy apparatus (e.g., a small-tube biopsy device) as described herein. FIG. 1A shows the biopsy apparatus in an un-deployed configuration while FIG. 1B shows the apparatus in a deployed configuration.

FIGS. 1C-1D show another example of a biopsy apparatus similar to that shown in FIG. 1A-1B; FIG. 1C shows the biopsy collector portion of the biopsy apparatus in an un-deployed configuration within the outer member (outer cannula). FIG. 1D shows the biopsy apparatus of FIG. 1C in a partially deployed configuration, illustrating the column strength of the biopsy collector portion of the apparatus configured to allow the device to push out of the outer member, and including an inner lumen.

FIG. 1E schematically illustrates an example of a biopsy collector having a low braid angle between the transverse fibers forming the biopsy collector; the low braid angle may provide a greater column strength and radial force.

FIG. 1F schematically illustrates an example of a biopsy collector having knit formed with a high needle count (e.g., between 22-50 needles) resulting in a more tightly packed knit having a high column strength and radial force.

FIGS. 2A-2F illustrate one method of using the apparatus of FIGS. 1A-1B to take a biopsy from within a body lumen.

FIG. 3A-3B is another example of a biopsy apparatus (e.g., a small-tube biopsy device) as described herein. FIG. 3A shows the biopsy apparatus in an un-deployed configuration while FIG. 3B shows the apparatus in a deployed configuration.

FIGS. 4A-4D illustrate one method of using the apparatus of FIGS. 3A-3B to take a biopsy from within a body lumen.

FIG. 5 schematically illustrates one example of a biopsy apparatus as described herein.

FIGS. 6A-6F illustrate the operation of a prototype biopsy apparatus as described herein.

FIGS. 7A-7E illustrate the use of a biopsy apparatus as described herein.

FIGS. 8A-8C illustrate an example of a biopsy apparatus as described herein including a proximal handle. FIG. 8A is a top view, and FIGS. 8B-8C show views.

FIGS. 9A-9B illustrate another example of a biopsy apparatus as described herein, in an un-deployed (FIG. 9A) and deployed (FIG. 9B) configuration, respectively.

FIGS. 10A-10B illustrate another example of a biopsy apparatus as described herein.

FIG. 11A schematically illustrates the use of a biopsy apparatus as described herein within a fallopian tube.

FIGS. 11B-11E illustrate the use of an apparatus as described herein to remove a clog or blockage within a body vessel, and/or to expand patency of the vessel. In this example, the vessel is a fallopian tube, however these methods and apparatuses may be used to unblock one or more other body lumen.

FIG. 12 schematically illustrates the use of a biopsy apparatus as described herein within a urethra and/or bladder.

FIGS. 13A and 13B illustrate an example of a biopsy apparatus including a biopsy collection portion that has a sleeve or liner. In FIG. 13A the apparatus is shown in a fully deployed configuration with the liner attached as part of the biopsy collection portion that also includes an outer, potentially more abrasive, member. FIG. 13B shows the apparatus with the biopsy collection portion partially retracted into the outer member.

FIGS. 14A-14C illustrate another example of a biopsy apparatus including a biopsy collector (collection portion) that is configured to jam in the un-inverted configuration to provide a minimum jammed diameter, forming a lumen through which drugs (e.g., including contrast), and/or one or more accessory tools may be passed. FIG. 14A shows the apparatus in a primarily un-deployed configuration, while FIG. 14B shows the apparatus in a deployed configuration. FIG. 14C shows the apparatus passing an accessory tool (e.g., camera) through the lumen formed by both the inner pusher catheter and the un-inverted portion of the biopsy collector.

FIG. 15A shows an example of a model of a female reproductive tract into which a prototype biopsy apparatus has been deployed. FIG. 15B illustrates one example of a prototype apparatus as described herein used to take a biopsy of an animal uterus (fetal bovine uterus). FIG. 15B is an image of a cells collected from the experiment shown in FIG. 15B.

FIG. 15C shows an example of bovine cells collected (and stained) at 100× magnification, from a preparation such as that illustrated in FIG. 15B.

FIGS. 16A-16B illustrate one example of an apparatus for performing a biopsy that includes a woven (e.g., braided) textile material that is configured to provide sufficient column strength and stability for the tube of textile material within the outer member (e.g., cannula) so that it does not buckle and/or expand outwards against the walls of the outer member, which may otherwise cause it to jam. FIG. 16A shows a schematic of the apparatus, while FIG. 16B illustrates the braid angle contributing to the column strength.

FIGS. 17A and 17B illustrate a first potential failure mode for an apparatus for performing a biopsy that includes a woven (e.g., braided) textile material. In FIG. 17A the tube of braided textile material has a braid angle that is greater than a threshold (e.g., 25 degrees). FIG. 17B shows the apparatus of FIG. 17A under compression, in which the inner member is advanced distally to extend and invert the tube of woven material from the un-deployed configuration to a deployed configuration. In FIG. 17B, the tube of braided textile material is shown expanding an locking against the walls of the outer member.

FIGS. 18A and 18B illustrate a second potential failure mode for an apparatus for performing a biopsy that includes a woven (e.g., braided) textile material. In FIG. 18A the tube of braided material has a small braid angle (e.g., 25 degrees or less) and a relatively large column strength under compression, but has an outer diameter of less than 85% of the inner diameter of the outer member. FIG. 18B shows the tube of braided material buckling within the outer member as the inner member is advanced distally.

FIGS. 19A-19C illustrate variations of biopsy apparatuses as described herein having different for performing a biopsy that includes a woven (e.g., braided) textile material.

FIG. 20 illustrates another example of a biopsy apparatuses having a tube of braided textile material with a high braid angle (e.g., 125 degrees or more) on the inverted portion of the braided tube that is extending distally from the outer member.

### DETAILED DESCRIPTION

Described herein are method and apparatuses (e.g., devices and systems) for taking a biopsy from the inside of a small tube without rupturing or dissecting the small tube. The methods and apparatuses described herein may be used for any body lumen, including in particular small-diameter body lumen, such as the fallopian tubes, urethra, etc.

In general, these devices may include a first end of a biopsy collector (e.g., biopsy collection member) connected at a first end (e.g., a distal end) to an inner member. The biopsy collector may be expandable, so that it may expand against the walls of the body lumen from which the biopsy is being taken. The second end (e.g., a proximal end) of the biopsy collector may be coupled to an outer member. This coupling may be rigid (e.g., fixed attachment) or movable (e.g., sliding, rotating, etc.). The inner member may be slideable within the outer member. The inner and outer members may be catheters, or other flexible, tubular members. In some variations the inner or outer members may be coils. The outer member may be coaxially arranged around the inner member. The device may be deployed by relative motion between the inner member and the outer member that allows the biopsy collector to expand to contact the walls of the lumen (e.g., of the urethra, fallopian tubes, etc.). For example, in some variations the inner member may be moved distally to allow the biopsy collector to expand out and in to the narrow tube (e.g., fallopian tube) to contact the walls and take a sample of the tissue; reversing the motion, the biopsy collector may be collapsed back down and in some variations drawn into the outer member, which may be a catheter. Any of these devices may also include a second outer member (e.g., outer catheter, such as a delivery catheter) that may house the inner and/or outer catheter to prevent loss or contamination of the biopsy material when inserting or removing the device.

In some variations the apparatuses described herein include an everting knitted or woven member for capturing tissue material, e.g., biopsy material, that forms all or a part of the biopsy collector portion of the device. The everting knitted or woven material may generally be adapted for use as a biopsy collection material. For example the everting knitted or woven material may include a column strength that is sufficient so that pushing on the material distally by pushing on the inner member drives the material out of the distal end of the outer material, allowing it to invert and expand against the inner lumen of the body vessel.

The biopsy collector material may be woven or knitted, as described herein, and may also be configured to remove material (biopsy material) from the lumen wall without puncturing or otherwise rupturing the lumen wall, but may also be configured to retain all or the majority of the tissue removed.

### Rolling Tube Biopsy Device

A small-tube biopsy device as described herein may have a biopsy collector that everts to sample the inner wall surface of a body vessel. The everting biopsy collector may therefore gently roll out into a small vessel or conduit to form a torus shape in which the outer walls of the torus are driven against the wall(s) of the vessel. When the biopsy collector is retracted back into the device, e.g., by inverting back into the device, the biopsy tissue captured by the biopsy collector may be protected.

In general, the rolling and everting motion of the biopsy collector may reduce or eliminate deployment shear force to the vessel/conduit wall. The variations described herein may therefore avoid hydraulic forces.

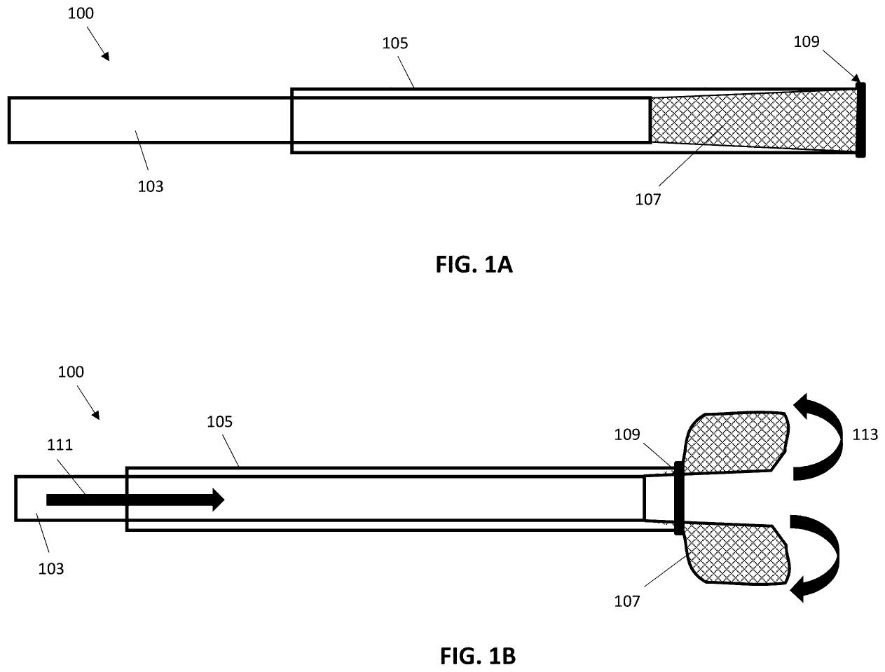

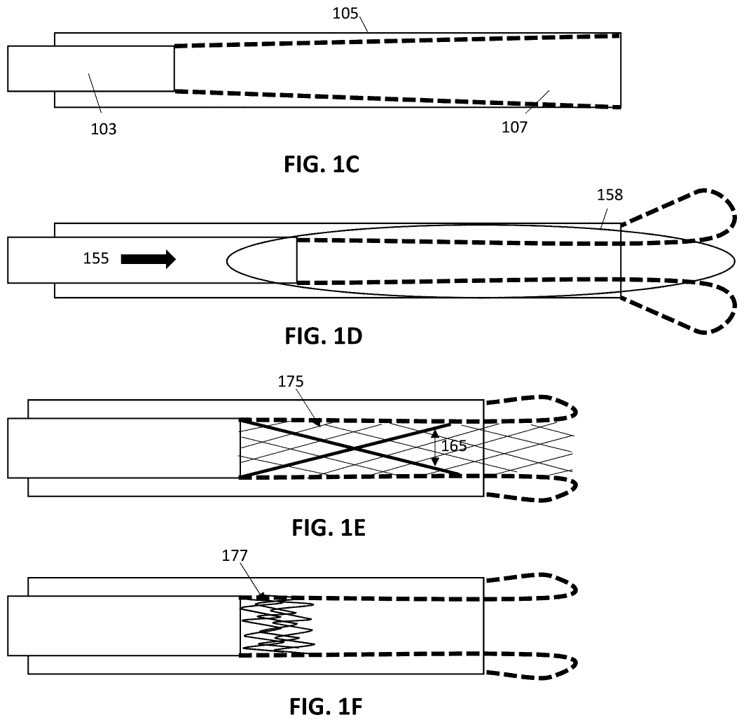

For example, FIGS. 1A-1B illustrate one variation of a small-tube biopsy device as described herein. In FIG. 1A, the biopsy device 100 includes an inner member 103 (e.g., inner pusher) that may be a rod, catheter, coil, etc. The inner member is typically flexible and may be hollow, which may provide a channel or passage for a guidewire. The inner member is coaxially arranged within an outer member 105 (e.g., outer sheath). The outer member may also be flexible and hollow, and may be a coil, catheter, etc. The inner member may move axially in a distal and proximal direction relative to the outer member. In FIG. 1A, distal is to the right, while proximal is to the left.

A biopsy collector 107 is attached at either of its ends to the inner member and the outer member in the example shown in FIG. 1A. The biopsy collector may include (or may consist of) a knitted or woven material, such as a knitted or woven textile material and may be configured to collect biopsy tissue. Alternatively, in some variations an additional material (e.g., a fabric or other material) may be formed as a sleeve, lining or other material that may be configured to collect and/or hold the biopsy material. The biopsy collector may be an everting member that, as shown in FIG. 1B is configured to expand when driven distally out of the outer member.

In FIG. 1A, the biopsy collector is shown in an un-deployed configuration, the majority of which (e.g., almost all, e.g., >95%) is housed within the outer lumen. The second (e.g., distal) end of the biopsy collector may be attached to the end region of the outer member, either within the inner lumen of the outer member or just over the edge (outer edge) of the outer member. In this example, the distal end of the outer member includes a soft bumper that may also or alternately be configured as a marker band for imaging, such as a radiopaque material. In some variations the distal bumper may be attached over the biopsy collector to secure it to the end.

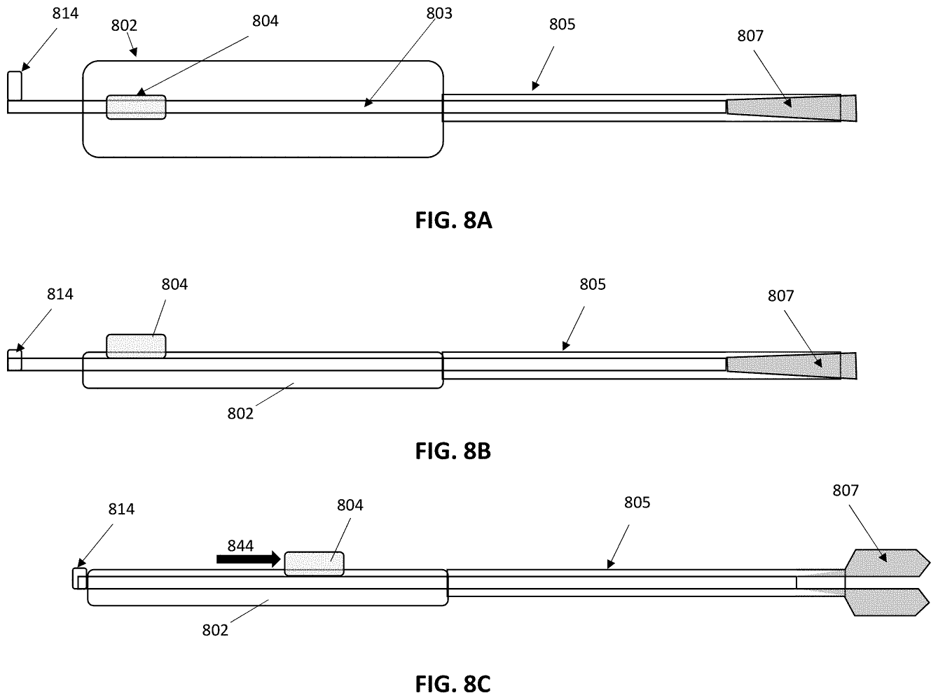

Having the biopsy collector retracted into the outer catheter as shown may protect the biopsy collector to prevent contamination as the device is positioned (e.g., over a guidewire) within the body. This configuration also forms a passage or channel for a guidewire. In some variations a separate guidewire lumen may be included within the inner member and/or the biopsy collector, e.g., by a sleeve or internal catheter, to prevent or limit contact between the biopsy collector and the guidewire. The device may also include a proximal handle (not shown in FIGS. 1A-1B, but described in FIGS. 8A-8D, below.

FIG. 1B shows the small-tube biopsy device 100 of FIG. lA in a deployed configuration. In this example, the inner member 103 has been advanced 111 distally, driving the biopsy collector 107 distally out of the end of the outer member 105, allowing it to evert 113 to form a torus shape, as shown. The biopsy collector is rolled out without sliding against the distal end bumper 109 of the outer member. The biopsy collector may contact the inner wall(s) of the body vessel, and may collect biopsy material, such as tissues or cells. In some variations the biopsy collector is configured so that it forms a rough surface to abrade or scrape the inner lumen of the body vessel. Alternatively, in some variations very little abrasive force is needed to sample the cells and tissue of the inner lumen. In this configuration, the apparatus may evert and compress the inner portion of the biopsy collector; this compression may prevent it from collapsing inward, and may allow it to stack up upon itself, so that the distal end rolls over itself to form the outer portion, as shown. In FIGS. 1A-1B the length is shown to be somewhat short, however, in practice the biopsy collector portion of any of these apparatuses may be configured to have a length that is between about 1 mm and about 15 cm long (e.g., between about 5 mm and about 12.5 cm, between about 1 cm and about 10 cm, etc.). The width of the biopsy collection portion of any of these apparatuses may be between about 0.5 mm about abut 9 cm (e.g., between about 0.5 mm and about 5 cm, between about 0.5 mm and about 2 cm, between about 0.5 mm and about 1 cm, etc.).

Thus, in use, no hydraulic forces are used, only everting column force. The biopsy collector may be any appropriate material. For example, in some variations the biopsy collector includes a laser slotted material, a textile (e.g., a braid, knit, or woven) material formed of filaments, or the like.

FIG. 1C shows another example of a small-tube biopsy device as described above. In this example, the biopsy device includes the outer member 105, shown as an outer catheter, and an inner member (inner catheter) 103. A flexible biopsy collector 107 is attached at one end to the inner member and at the opposite end to the outer member. In this example the flexible biopsy collector is attached to an inner surface of the outer member or alternatively at the annular lip of the outer member. The biopsy collector may be referred to as a textile in this example as it may be formed of a material that is woven, knitted or braided. The biopsy collector in FIG., 1C is shown in a pre-eversion configuration and includes an inner lumen or passage that may pass a guidewire, guide cannula or another catheter. In some variations a fluid (water, dye, etc.) may be passed through the inner lumen.

In general the biopsy collector may be configured so that, when pushed distally 155 by the inner member, it has sufficient column strength so that it can push the textile material out of the distal end of the outer member, as shown in FIG. 1D. For example, the biopsy collector may stack up so to have a greater column strength in the un-everted portion (shown in the circled region 158).

The column strength may be set by the structure of the biopsy collector. For example, FIG. 1E illustrate an example of a biopsy collector formed as a weave or braid 175 having a low braid angle when compressed. The braid angle 165 is the distal-facing angle between overlapping strands (e.g., filaments). In some variations the low braid angle may correspond to a braid angle between each wire in this distal-facing direction of between about 5 degrees to about 45 degrees, (e.g., between 5 and 40 degrees, between 5 and 30 degrees, between 10 and 25 degrees, between 15 and 25 degrees, about 15 degrees, about 20 degrees, less than about 40 degrees, less than about 35 degrees, less than about 30 degrees, less than about 25 degrees, etc.). Note that in some variations the biopsy collector may be woven and the weave may have a braid angle that is zero, as all warp yarns may be in line with the long axis of the outer member (e.g. outer catheter).

FIG. 1F shows an example of a knitted version of a biopsy collector 177 (only a representative portion of the knit is shown). In particular, knitted biopsy collectors (textile biopsy collectors) may have a high column strength when the knit has a high needle count, e.g., between about 22-50 needles (between about 20-60 needles, between about 22-50 needles, between about 25-45 needles, etc.), and forms a short stitch/loop (e.g., about 3 mm or less, about 2.5 mm or less, about 2 mm or less, about 1.8 mm or less, about 1.5 mm or less, etc.). The knit may be tightly packed length-wise, making the compressed weave more dense/tight, which may increase column force.

As mentioned, in any of the biopsy collectors described herein the biopsy collector may be formed by a combination of materials, including metallic materials (e.g., shape memory alloys, such as Nitinol, Steel, Elgiloy, MP35n, etc.) that may be filaments or wires, and one or more polymeric filaments or natural filaments. These filaments may be monofilaments or groups of filaments. In some variations the ratio of metallic filaments to non-metallic filaments (e.g., polymeric filaments) may be 1:1 or may have more non-metallic filaments, such as 1:2, 1:3, 1:4, 1:5, etc. Ins some variations there may be more metallic than non-metallic filaments, e.g., the ratio of metallic filaments to non-metallic filaments may be, e.g., 2:1, 3:1, 4:1, 5:1, 6:1, etc.

The non-metallic filaments may help in capture and retaining the biopsy material; in some variations the metallic filaments may help in removing the biopsy material when pulled against the wall of the vessel.

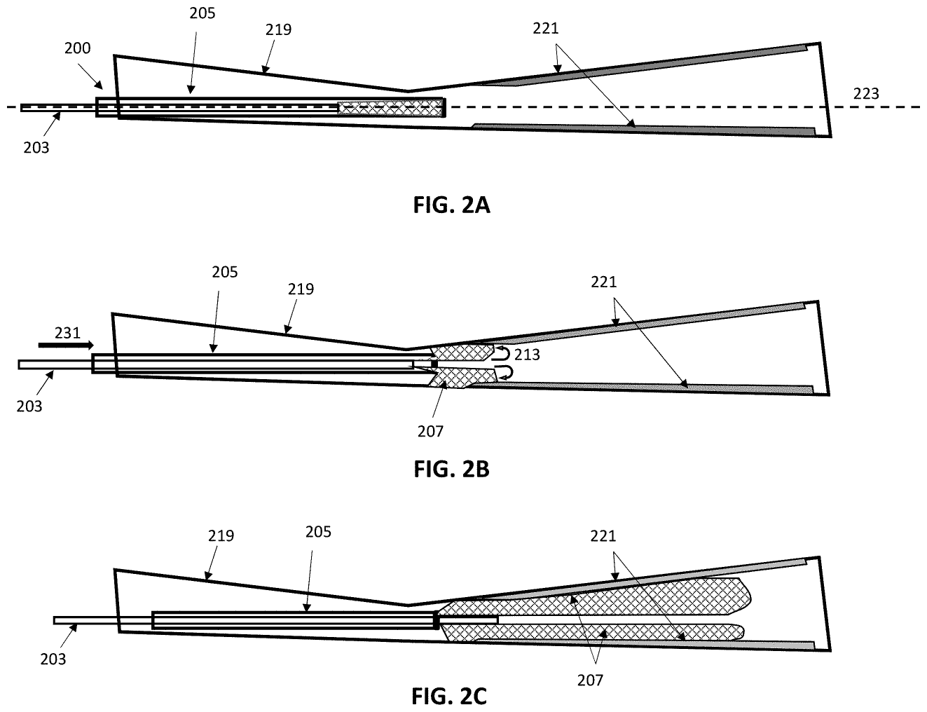

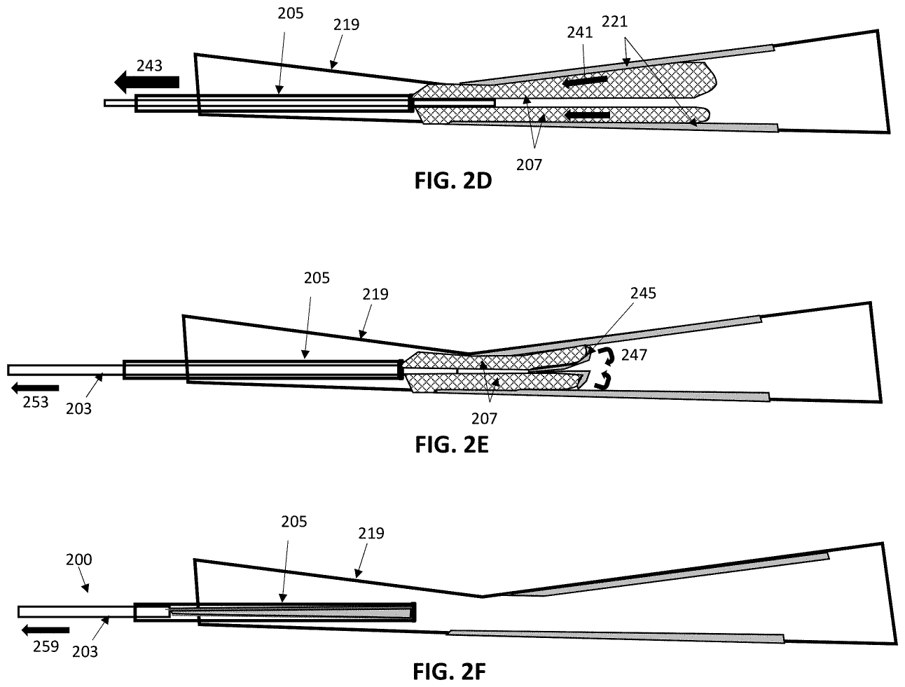

As mentioned above, once the biopsy collector is deployed, a pulling shear force may be applied to the structure to gently scrape the vessel/conduit wall to take a tissue sample/cells from the inner wall. This is described in FIGS. 2A-2F, illustrating the operation of an exemplary device such as the one shown in FIGS. 1A-1B. In this example, as shown in FIG. 2A, the device 200 is inserted into a vessel 219. The vessel, such as a fallopian tube, may have a variable diameter, and a portion of the vessel may include a region from which a biopsy is to be taken (shown in FIG. 2A as a region of interest in the tissue 221).

In this example a guidewire 223 may be first directed in to the vessel to or past the region to be biopsied 221. The small-tube biopsy device may then be sent over the guidewire in the un-deployed configuration, as shown in FIG. 2A, with the biopsy collector (comprising a knitted or woven textile) retracted into the outer member and with a first end of the biopsy collector connected to the distal end of the inner member and the second end of the biopsy collector connected to the distal end of the outer member. Once the device is positioned with the distal end of the outer member at or near the region to be biopsied (such as, for a fallopian tube, at the entrance into the fallopian tube), the inner member may be pushed distally 231, as shown in FIG. 2B. This force may drive the biopsy collector 207 out of the distal end of the outer member without rolling over the distal end of the outer member. The guidewire may be left in place or removed (not shown in FIGS. 2B-2F).

Thus, the method of using the device may include moving the inner member of the biopsy device distally within the outer member of the biopsy device so that the biopsy collector (e.g., the knitted or woven textile forming at least part of the biopsy collector) is everted 213 as a torus along the inner surface of the vessel 219. The biopsy collector remains attached to a distal region of the inner member 203 and to a distal region of the outer member 207 so that movement of the inner member causes the biopsy collector to roll out of a distal end of the outer member and expand against an inner wall of the body lumen, as shown in FIGS. 2B-2C. This process may be performed with minimal or no shear on the vessel wall.

In some variations, the biopsy collector may then be moved (e.g., moving the knitted or woven textile) relative to the inner wall of the body lumen to capture cellular material from the body lumen on the knitted or woven textile. This movement may include scraping the biopsy collector against the lumen wall, e.g., by rotating, pulling or pushing the biopsy collector relative to the wall, or it may simply include allowing the biopsy collector to roll against the lumen wall when deploying the biopsy collector, as shown in FIG. 2C.

FIG. 2D shows the biopsy collector being drawn against the lumen wall by pulling the device proximally to pull the knitted or woven textile of the biopsy collector along 241 the wall of the lumen. This may be done, as in this example, by securing the inner and outer members relative to each other so that they move together, and pulling them both proximally 243, as shown. As a result a biopsy sample (e.g., cell or tissue sample 245) may be retained on the biopsy collector, as shown in FIG. 2E.

In FIG. 2E the biopsy collector may then be retracted back into the outer member with at the biopsy material 245 attached. The gentle rolling motion 247 of the biopsy collector being retracted by pulling 253 the inner member proximally may prevent dislodging the biopsy material from the biopsy collector, further once within the outer member the biopsy material may be protected from contamination and dislodgement, as shown in FIG. 2F. The entire device 200 may then be withdrawn proximally 259, as shown in FIG. 2F.

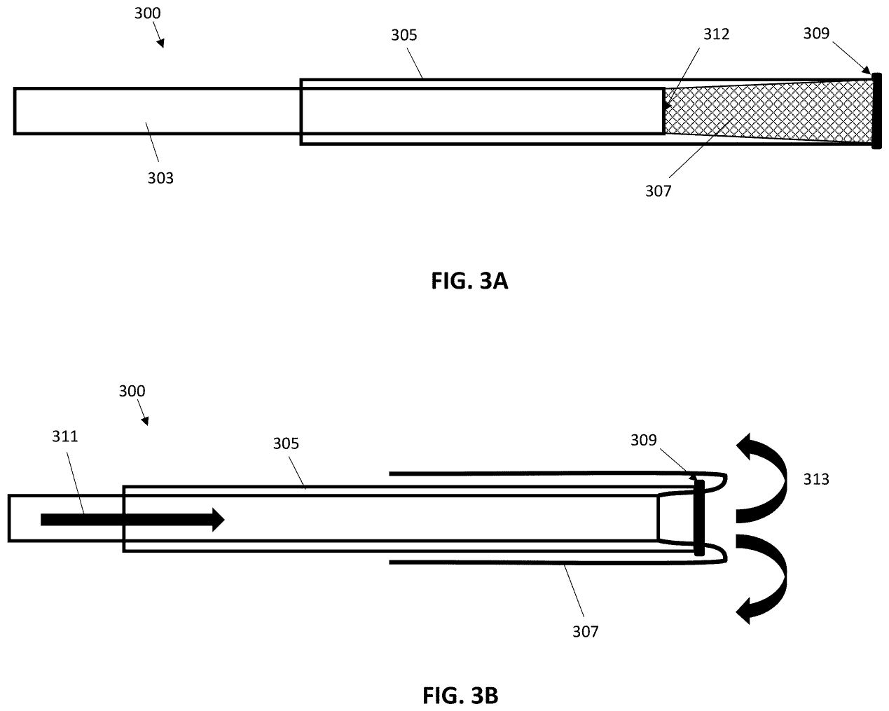

FIGS. 3A-3B illustrate another example of a small-tube biopsy device as described herein. In this variation, the biopsy collector may be attached at the proximal end to the inner member, but may optionally be unattached at the second, distal end to the outer member. In FIG. 3A the device 300 includes an inner member 303 that is coaxially arranged within the outer member 307. The biopsy collector 307 (which may include a knitted or woven textile) that is configured to collect biopsy tissue is attached at one end to the distal end of the inner member 312. FIG. 3A shows the un-deployed configuration. FIG. 3B shows an example of the deployed configuration in which the biopsy collector is driven distally by pushing distally 311 on the inner member, as shown. The outer member may also include a distal tip region 309 that is configured as a soft tip/bumper and/or marker. FIG. 3B, the distal tip may be positioned near the biopsy region or just past it, and the inner pusher driven distally to cause the biopsy collector to extend out of the distal end and evert and roll back 313, as shown. In this example, the everting and rolling of the biopsy collector occurs past the distal tip of the outer member and is also prevented from rolling on or over the distal tip, as shown. Thus, the everting biopsy collector turns inside out to create the everting rolling member (the biopsy collector 307).

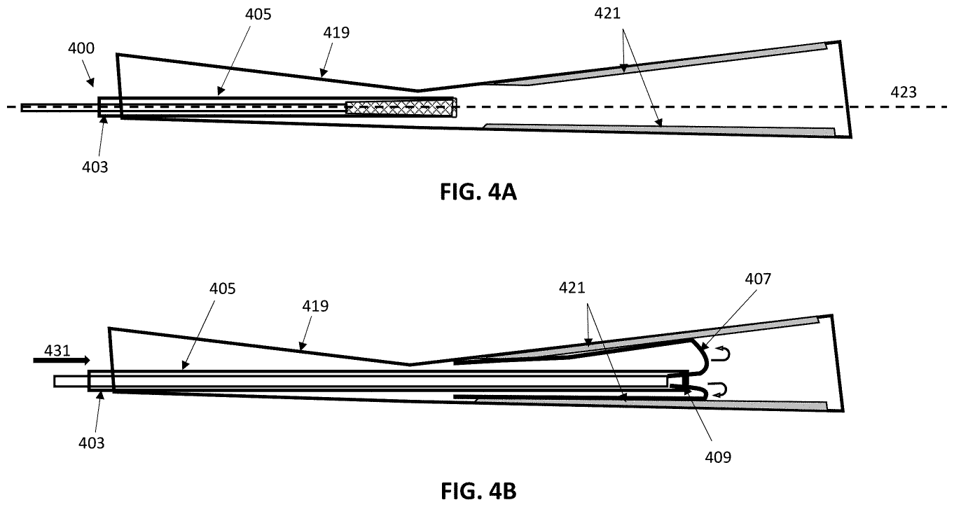

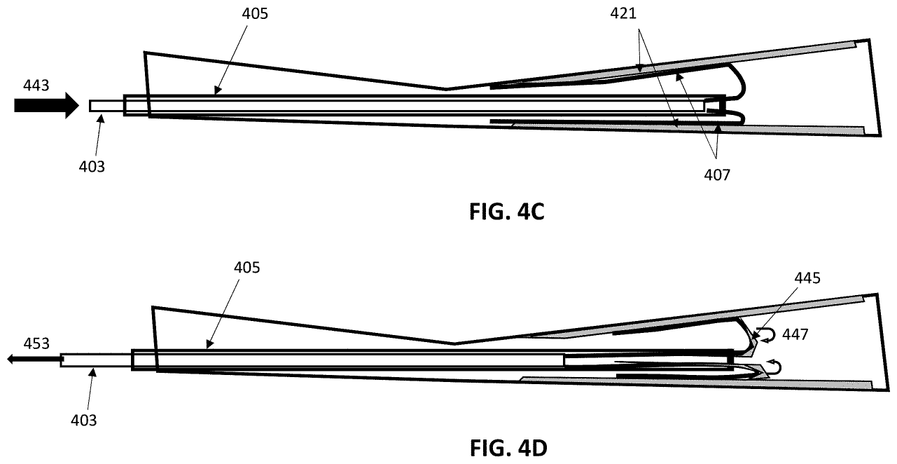

FIGS. 4A-4D illustrate one example of a method of taking a biopsy from within the lumen of a vessel using the device of FIG. 3A-3B. In FIG. 4A, a guidewire 423 may be first positioned to the target region 421 and the device 400 may be moved into position over the guidewire as shown. One in positon, the inner member 403 may be pushed 431 distally while holding the outer member 405 fixed, as shown in FIG. 4B. This will deploy the biopsy collector (e.g., knitted or woven textile) 407 so that it everts and rolls back, without rolling against the distal opening 409 of the outer member, as shown. As it is deployed, the biopsy collector may be deployed against the inner lumen of the vessel 419, as shown, and in particular against the target tissue region 421 within the lumen of the vessel. There may be very little, if any, shear between the everting member 407 and the vessel wall, preventing dislodgement of the tissue.

The entire device may then be moved 443 relative to the vessel, as shown in FIG. 4C, to scrape the biopsy member against the target tissue region to take the biopsy sample for retention in the biopsy collector 407 (e.g., the knitted or woven textile and/or a sleeve or other retention material). Thereafter the biopsy collector 407 may be retracted back into the outer member by pulling 453 the inner member proximally, as shown in FIG. 4D. The biopsy collector may retain and remove the biopsy material 445 as it is rolled 447 back into the outer member 405. Withdrawing the biopsy collector may also shear the biopsy material from the vessel, as shown.

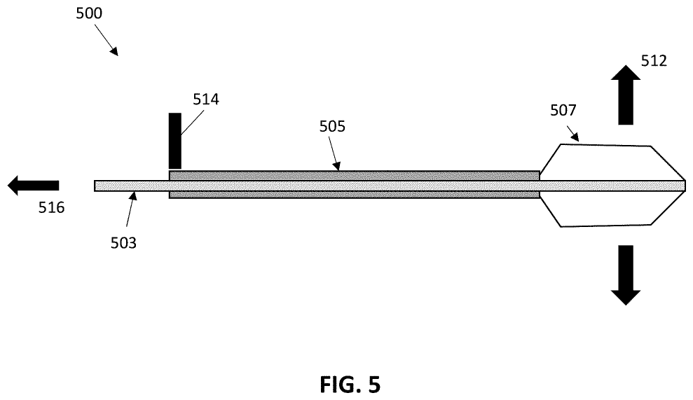

As illustrated schematically in FIG. 5, there are various features of the devices described herein, and in particular of the biopsy collector, that may affect the ability of the device to evert, collect, and retain biopsy material. For example, the biopsy collector, and in particular the knitted or woven textile forming a surface of the biopsy collector, may have a surface texture that may be rougher or softer. In general, rougher surfaces may gather/shear more tissue. For example, the filaments used to form the biopsy collector may be, e.g., wires or strands that have a rounded, flat or other geometric shaped cross-section (e.g., triangular, rectangular, etc.). For example the biopsy collector may be formed at least in part of woven, braided or knitted wires that have wire shapes that may be rounded vs. flat.

As mentioned above the biopsy collector may have a porosity that is configured to help with retention of biopsy material. For example the apparatus may have pores of bigger or smaller gaps; bigger gaps may be formed from using fewer filaments (e.g., less wires per unit circumference), which may also increase roughness. The material used to form all or a portion of the biopsy collector may be metallic, polymeric (e.g., smooth and/or hairy elements), natural fibers, etc.

The biopsy collector may also be configured to adjust the radial force or contact force with the lumen of the vessel. For example, a greater radial force may increase the shear. The filament diameter (e.g., wire diameter), the filament material (e.g., metallic, polymeric, natural, etc.), and/or the number of filaments forming the biopsy collector may all contribute to the radial force as well as the ability to capture biopsy material. In general, the mechanical leverage provided by the biopsy collector may be related, at least in part, to the radial force that can be increased by the proximal handle, which may create an increased radial force by using the inner and outer members (e.g., inner and outer catheters). Any of the biopsy collectors described herein may also be formed of multiple layers. For example, a rougher outer layer (e.g., a rougher woven/knitted outer portion of the biopsy collector) may be adjacent to a more tissue-retaining layer that may be ‘softer’. For example, a woven or knitted material that is formed of a natural fiber or other material configured to retain tissue.

The length of the biopsy collector may also be adjusted. For example, the length of the biopsy collector may be adjusted to adjust the applied force per unit length applied to the vessel. Shorter lengths (e.g., lengths of between 0.2 cm to 50 cm, e.g., between 0.2 cm to 30 cm, between about 0.2 cm to 25 cm, between about 0.2 cm to 20 cm, between about 0.2 cm to about 15 cm, between about 0.2 cm to 10 cm, etc.) may be used.

In general, the biopsy collector (which may alternatively be referred to herein as an everting biopsy collector) may also be pre-shaped or biased to a particular form, such as a self-expanding configuration, as mentioned above. For example, the biopsy collector may be annealed into an expanded configuration. In any of these variations, the distal member may be pre-shaped (e.g., annealed) and/or reinforced to deploy the biopsy collector to a set predefined length, so that the distal end and proximal end are pre-shaped into a deployed configuration when extended distally, as shown in FIG. 5. In this example, the device 500 can be locked when deployed. In FIG. 5, the device includes the inner member 503, outer member 505 and the biopsy collector 507. The biopsy collector may be reinforced and/or pre-shaped to expand and may resist invagination. Reinforcement can be due to additional wires, lamination with polymer/skin, internal support structures. Thus, the biopsy collector may be biased 512 to expand outwards when deployed by driving the inner member distally and retaining the outer member in a fixed (relative) position) 514. The biopsy collector may be retracted by pulling the inner member 503 proximally 516. In addition, in the reinforced variation shown in FIG. 5, pulling back on the inner member with the outer member pinned as shown may result in an increased outward radial force.

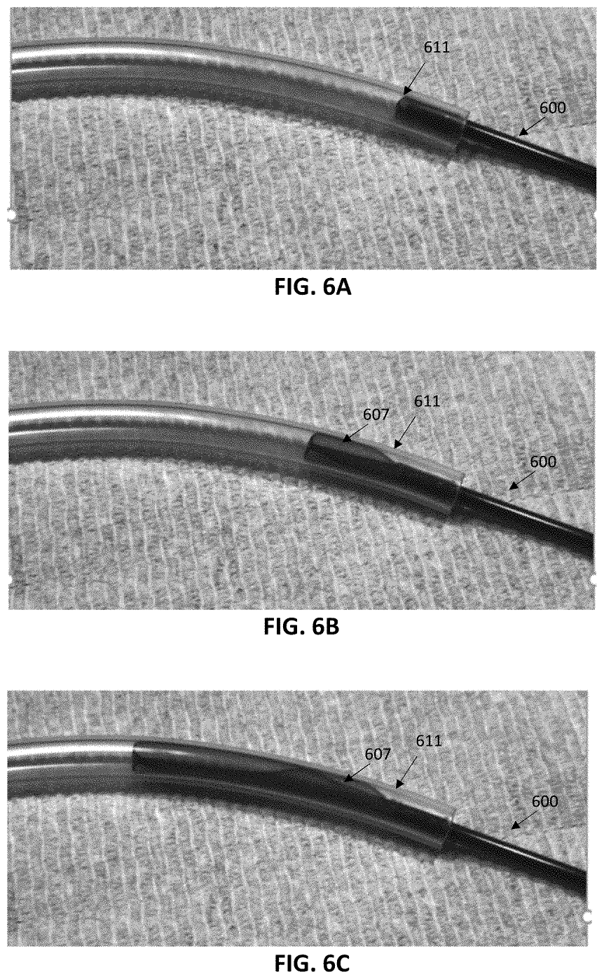

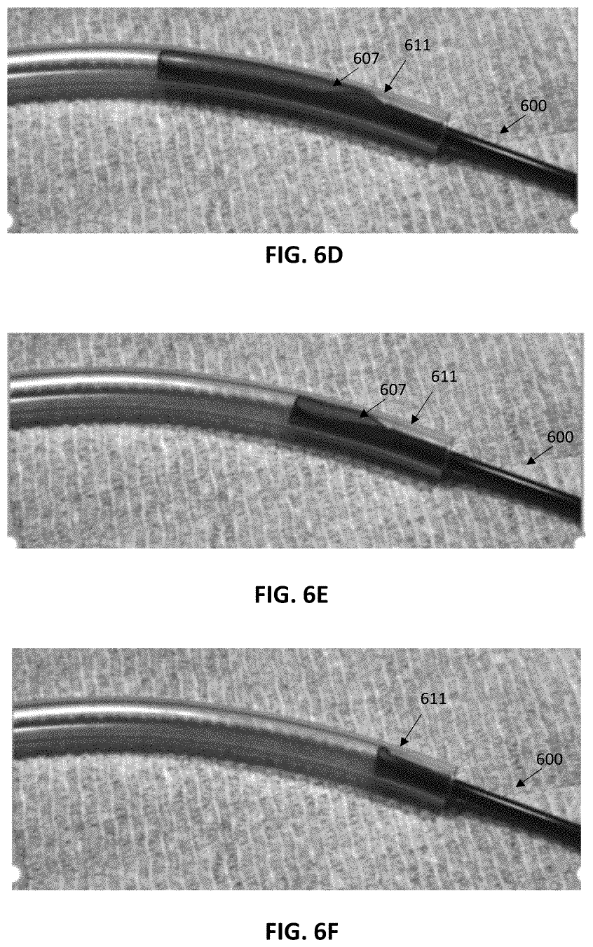

FIGS. 6A-6F illustrate a prototype of the apparatus of FIGS. 1A-1B within a model of a lumen. In FIG. 6A the device is positioned within the model lumen; the device is initially in an un-deployed configuration. In FIG. 6B the device is shown deploying by driving the biopsy collector 607 distally so that it unrolls against the inner lumen wall, as shown. In FIG. 6C the device is shown fully deployed, and may be moved relative to the lumen to collect biopsy material. FIGS. 6D-6F illustrate retraction of the biopsy collector by withdrawing the inner member (not shown) proximally to draw the biopsy collector 607 back into the outer member.

In some variations the devices described herein may include a biopsy collector that includes a laser-slotted tube, such as a tube having a 0.002″ thick wall (or a wall thickness of less than about 0.0002 to 0.0004″). The tube may be formed of a shape memory material, such as Nitinol (e.g., having a 1 mm OD). In some variations the biopsy collector comprises a textile that is braided and has between 24-72 ends (e.g., strands), having a 1 mm OD, and a low braid angle. The strands may be between about 0.00025″ to 0.002″ diameter, and may be round or flat wires, such as Nitinol, Platinum Iridium (which are radiopaque). In some variations the biopsy collector comprises a polymer metal hybrid that may include Nitinol mixed with PET multifilament (e.g., 0.001″ Nitinol with 100 Denier PET multifilament highly textures), e.g., braided to a 10 mm diameter in the jammed state (e.g., compressed within the outer member).

In any of these variations, twisting or rotating the inner and outer members relative to the everting member may create a cork screw like surface on everting member, increase tissue scraping effect.

In some variations the biopsy collector may be a knit material. The knit may be a circular or warp knit, for example, formed from 0.001″ Nitinol wires that are mixed with 100 denier PET multi filaments (which may result in a highly textured knit).

For example, a biopsy collector may be configured to expand when it is everted, as described above, which may help contact the wall(s) of the vessel lumen. In some variations the biopsy collector may be configured to expand uniformly, e.g., to a uniform expanded diameter. Alternatively, the biopsy collector may be configured to expand to a shape, such as a tapered shape. In general the expanded biopsy collector may be configured to roll over the inner lumen of the vessel even where the vessel wall is uneven.

In any of the biopsy collectors described herein the surface texture may be adjustable from smooth to rough. For example, the biopsy collector may be formed of fine, tight filaments, which may provide a smoother, less abrasive surface, or may be formed of larger, less round or flat filaments (flat wires, multifilament yarns) which may provide a rougher, more abrasive surface. As mentioned, the biopsy collectors described herein may also be configured to adjust the porosity. For example, biopsy collectors having fewer filaments may have an increased roughness, but higher porosity.

In any of the biopsy collectors described herein, the contact force of the biopsy collector as it expands into the vessel may be adjustable (and in particularly, non-hydraulically adjustable). The contact force of the biopsy collector may adjustable to the vessel/conduit inner diameter (ID). This contact force may be adjusted by selecting the filament size, filament material type, and/or by adjusting the relative positions of the inner member to the outer member position. As described above in FIG. 5, this may adjust the everting member expansion force.

Any of the variations described herein may be configured to allow visualization during use, including with fluoroscopy or other techniques. For example, the material used for any part of the devices described herein, including the biopsy collector, inner member, outer member, etc. may be formed of or marked with a material, e.g., polymer, metal, type of metal and size of metal, etc., including mixes of radiopaque materials and other materials. For example, the biopsy collector may include a radiopaque material and a tissue grapping material (e.g., a combination with a polymer such as PET).

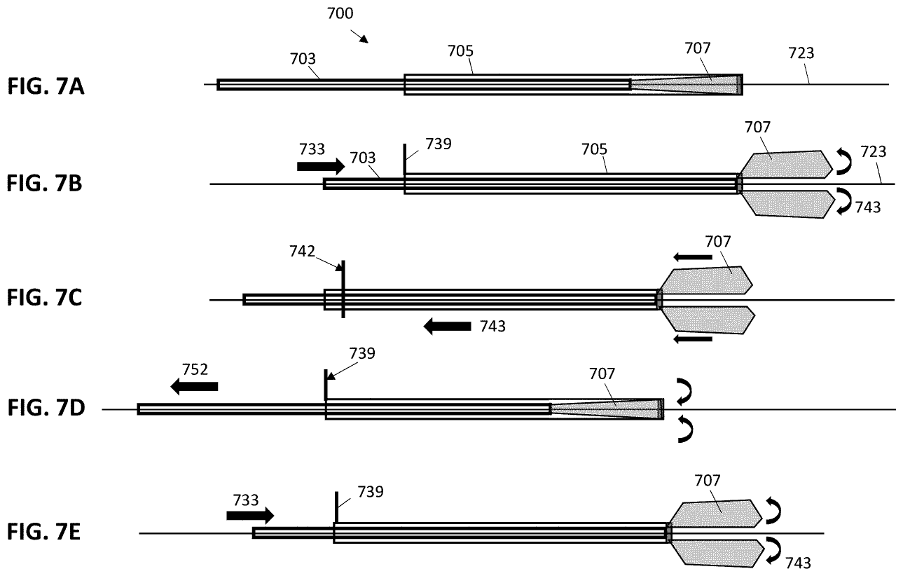

FIGS. 7A-7E illustrate another example of a method of using a device such as the one shown in FIGS. 1A-1B. In FIG. 7A the device, which includes an inner member (inner catheter) 703, and outer catheter 705, and a biopsy collector 707, is shown in an un-deployed configuration passing over a guide wire 723. The tip of the device 700 may be advanced to the mouth of a vessel of an organ of interest, e.g., through a guide catheter or cytoscope, using a marker band, fluoro and direct visualization through fiber scope assure position, with our without guide wire 723. Once the tip of the device is in position, the outer member may be fixed in place 739 (e.g., pinned, at an anchor position), and the proximal end of the inner member may be pushed/advanced 733, so that the inner member that is coaxial within the outer member is driven forward, which may extend the biopsy collector distally so that it everts and begins unrolling 743, as shown in FIG. 7B. The inner member may be advanced as much as desired, such as for a short length or for the full length of the tissue sampling region.

FIG. 7C illustrates one method of taking a sample. Once the biopsy collector, in this example, formed of a knitted or woven material, has been everted and much as desired, it may be moved relative to the lumen of the vessel to collect sample material. For example, the inner and outer members may be coupled (e.g. pinned) together 742 and the entire device may be moved proximally so that the textile of the biopsy collector drags across the tissue and may take the sample. For example, the entire device may be moved slowly proximally to drag the entire system backwards about 1-10 cms; this length may depend on the amount of tissue sample desired.

The biopsy sample may then be recovered as illustrated in FIG. 7D. In this example, the outer member may be held in a fixed position 739 (e.g. pinned) while the inner member is moved proximally 752 until the everting biopsy collector (e.g., textile element 707) is fully recaptured within the outer member, as shown. The inner and outer members may then be pinned together (e.g., moved together) to withdraw the entire device from a guide or cystoscope (e.g., a guide catheter, not shown), including removal over a guidewire if used.

Once removed from the body, the biopsy sample may be tested in any appropriate manner. For example, FIG. 7E illustrates removal of the biopsy material from the device for further processing, including for histology. For example, to collect the tissue for histology, the inner member may be advanced relative to the outer member to evert the biopsy collector (e.g., textile) and biopsy collector may be rinsed in an appropriate fluid collection vial, e.g., containing a buffer.

As mentioned above, any of the apparatuses described herein may include proximal handle that may coordinate the operation of the device. For example, FIGS. 8A-8C illustrate one example of a device including a proximal handle; a similar proximal handle may be used with any of the devices described herein. In FIG. 8A a top view of the proximal handle 802 shows a control 804 that is connected to the inner member 803; the outer member 805 is connected to a housing portion of the proximal handle 802. The biopsy collector 807 is attached to the distal end of the inner member, and is shown in an un-deployed (e.g., pre-deployed) configuration. In this example, the control 804 is shown as a slider, so that advancing or retracting the slider proximally/distally will proximally or distally advance/retract the inner member and therefore the biopsy collector. In some variations the control is a deployment/recapture sliding button, button on ratchet (e.g., in which each click is some amount of deployment distally). The device may include a flush port 814 for coupling to a flushing fluid (e.g., in a syringe, etc.) that may pass through the inner lumen of the inner member. FIG. 8B shows a side view of the device of FIG. 8A. FIG. 8C is another side view of the apparatus of FIG .8B, showing deploying the biopsy collector 807 by operating the hand-held control on the proximal handle. In FIG. 8C the control is a slider that is advanced distally 844 to deploy the biopsy collector as described above. FIG. 8

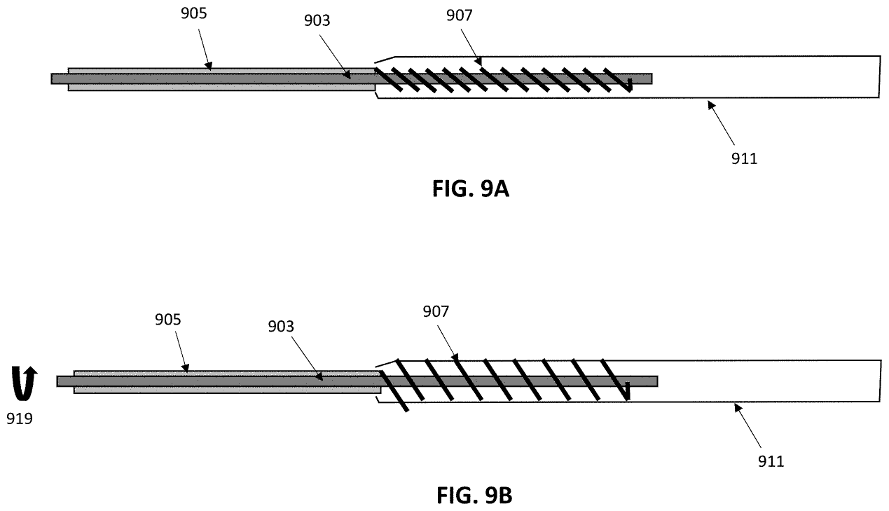

Another example of a small-tube biopsy device in which the biopsy collector is configured as a helically wound coil is shown in FIGS. 9A-9B. In this example the device 900 includes an inner member 703, an outer member 905 and the inner member is coaxially arranged within the outer member, so that the inner member may move relative to the outer member. In this example, the biopsy collector is configured to collect biopsy tissue includes a coil 907 that is helically wound around the inner member. The biopsy collector is connected at a first end to the inner member and at a second end to the outer member. In this example the device is shown inserted into a body lumen, such as a fallopian tube 911. In FIG. 9A-9B, the coil is connected at either end to the inner and outer members. Inner member could be wire or catheter. As shown in FIG. 9B, the biopsy collector may be expanded by rotating 919 the inner member while holding the outer member fixed (e.g., pinned), which results in opening and expanding of the coil 907, as shown. Alternatively, the coil may be biased to expand and released (unpinned) from the outer member to allow the inner member to rotate so that it may expand as shown. Once expanded, the device may be moved so that the biopsy collector (including the coil) is moved relative to the inner lumen to scrape against the tissue.

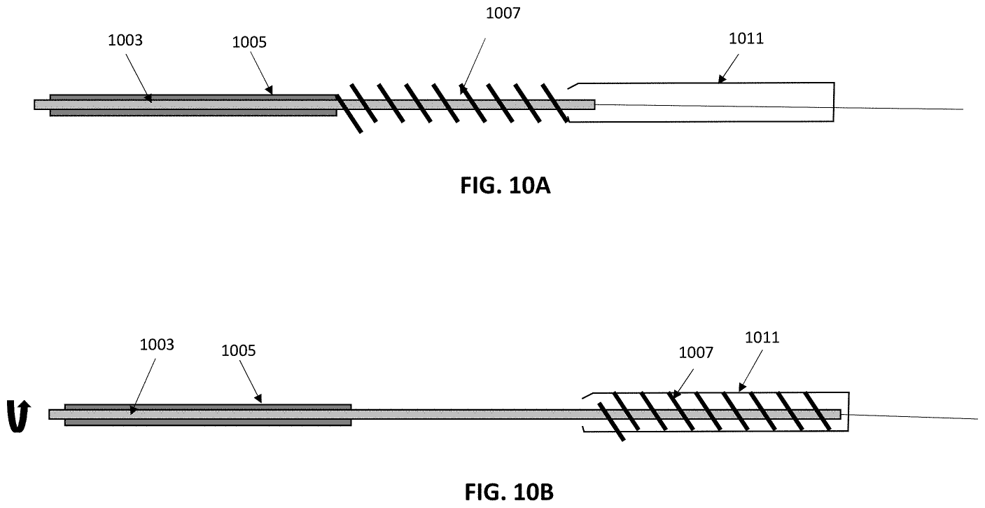

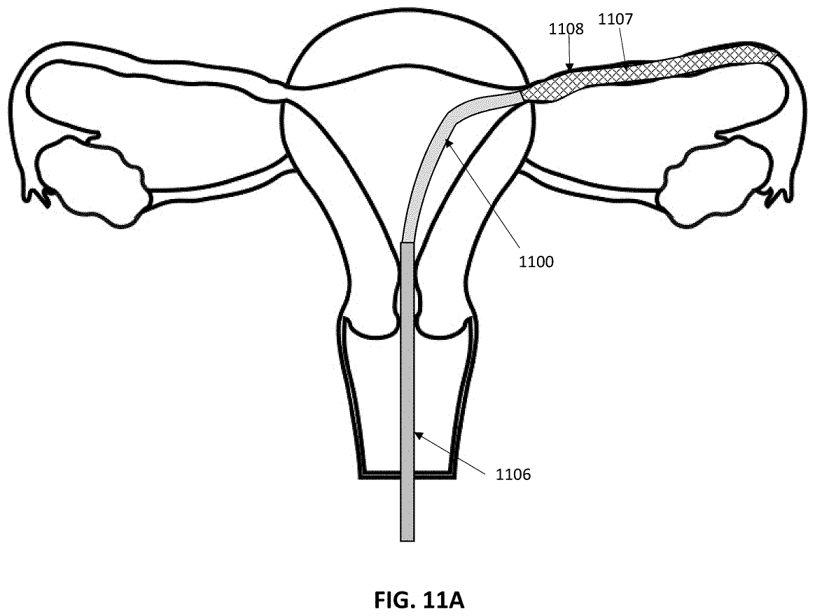

Another alternative variation is shown in FIGS. 10A-10B. In FIG. 10A the inner member 1003 includes a helically wound coli 1007 attached over the distal end region. The helically wound coil potion may be rotated relative to the outer member 1005 to screw the biopsy collector (including the coil) into the body lumen 1011, as shown in FIG. 10B.

In the variations shown in FIGS. 9A-9B and 10A-10B in which a coil is included, the coil may be, e.g., made from a flat Nitinol wire (e.g., 0.003″ by 0.008″) or a steel-like material (e.g., Elgiloy, MP35n, etc.) at 0.002″ by 0.008″. In the variations such as those shown in FIG. 9A-9B, in which the coil may be tightened down onto the inner member, the outer diameter of the tightened shape may be less than about 1 mm OD, while the un-twisted (e.g., release/expanded configuration) may be 1.5-2.0 mm. In some variations the length is approximately 8 cm long in total length (e.g., between 0.5 and 10 cm, between 0.5 and 9 cm, between 1 and 8 cm, between 1 and 7 cm, between 1 and 6 cm, etc.). In variations including a coil (e.g. stent or stent-like) configuration, the stent-like structure may have a 1 mm constrained diameter and an approximately 1.5-2 mm expanded diameter. The stent may be made from a material such as Nitinol.

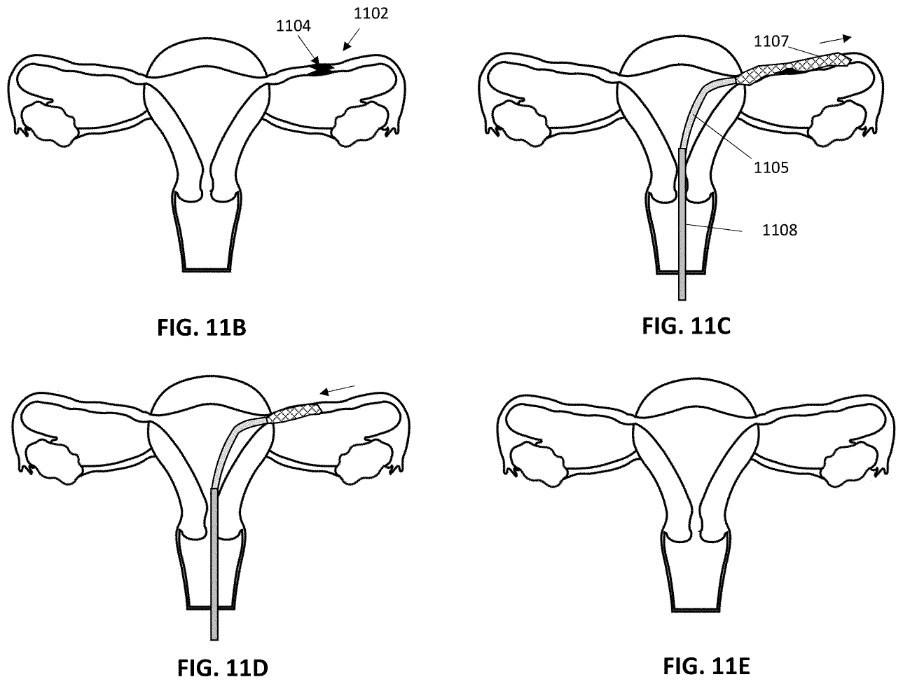

As mentioned above, the biopsy apparatuses described herein may be used with any appropriate body lumen, including a fallopian tube. This is illustrated in FIG. 11A, showing a biopsy apparatus as described above 1100 inserted through a guide catheter 1106 and into a fallopian tube 1108. The biopsy collector portion 1107 of the device is shown everted and taking a tissue sample from within the fallopian tube ID. This may aid in screening for ovarian cancer, for example.

In general, the fallopian tube may have an inner diameter that is less than about 1 cm, thus the apparatuses described herein may be configured so that the biopsy collector portion is annealed to have an outer (everted) diameter that is slightly larger than the inner dimeter (may be between 0.5 cm and 1.5 cm, between 0.7 cm and 1.2 cm, etc.). In some cases the inner lumen of the fallopian tube may have a somewhat irregular (e.g., non-smooth) surface in cross-section. The biopsy collectors described herein, and particularly those formed of a textile material (e.g., braided, woven and/or knitted) may be sufficiently compliant to allow expansion when everting and apply radially outward force against this irregular surface without shearing the inner lumen during deployment. In some cases the tubular body (including but not limited to the fallopian tube) into which the device is to be deployed may be configured so that it expands and opens the tubular body as it is deployed. As described in greater detail below, the apparatuses described herein may therefore be configured to open, and in some cases clear out, a vessel instead or in addition to biopsying the vessel.

Any of the devices, and particularly the biopsy collector portion of the device may be configured to provide a radial force when deploying that is sufficient to open and/or expand a tubular structure, such as fallopian tube. The radial force may be determined or adjusted by, for example, the stiffness of the filaments (e.g., wire and/or polymer or natural filaments) used to form the biopsy collector portion. Thus the hoop stiffness of the biopsy collector may be sufficient to hold open the vessel and/or to apply adequate radial force against the wall of the vessel so that a biopsy can be taken even when the wall has an irregular cross-section.

FIGS. 11B-11E illustrate the user of biopsy device as described herein to take a sample of a material. In FIG. 11B the fallopian tube 1102 includes a region that is to be sampled 1104; one or more other regions may also be sampled (e.g., along a portion or all of the fallopian tube). In FIG. 11C a biopsy device as described herein may be deployed in to the fallopian tube. For example, the system may include a delivery catheter 1108 that is used to deliver the biopsy device including an outer catheter 1105 and an inner catheter (not visible), and a biopsy collector, configured as a textile (woven, knitted or braided) material 1107. The device may be deployed over a guidewire or guide catheter (not shown) or without a guidewire or guide catheter. The device may be deployed over the region to be biopsied by pushing the biopsy collector distally out of the outer catheter so that it everts and rests against the inner surface(s) of the vessel lumen. In variations in which the vessel lumen has an irregular shape, the everting biopsy collector region may conform to the wall shape without applying shear force against the wall. The radial force may be sufficient to hold the device against the wall without harming the tissue. The device may be extended along a portion of the fallopian tube or the entire length of the fallopian tube, by everting the biopsy collector from the outer catheter. In some variations, the device may then be moved distally (pulled) and/or rotated, e.g., to scrape against the walls of the fallopian tube and the biopsy sample taken. In some variations scraping is not necessary. As shown in FIG. 11D, the biopsy collector region may then be withdrawn into the outer catheter by pulling the inner catheter proximally to invert the biopsy collector back into the outer catheter; the biopsy collector may be completely withdrawn into the outer catheter, and the device maybe removed (FIG. 11E). In some variations, the cervical region (cervix) may also be sampled using the same device.

Once collected, the sample may be stored and/or analyzed. For example, the biopsy collector region may be everted back out of the device and into a sample storage device, e.g., containing a buffer or other storage medium. In some variations the entire distal end of the device may be stored for later removal of the biopsy material from the biopsy collector region. Alternatively in some variations the biopsy collection region may be disengaged from all or a portion of the device and stored in a storage medium for later processing. For example, the distal end of the outer member and/or all or a portion of the inner member may be cut or otherwise removed from the rest of the apparatus and stored in buffer/storage medium. In some variations the device may include a detachment or frangible portion to allow detachment and separation of the distal end region including the biopsy collector portion to be removed for storage and later for removal of the biopsy material. In some variations the biopsy collector portion may be pushed out and cut off of the inner and/or outer members for insertion into a storage medium (e.g., within a tube) for later processing.

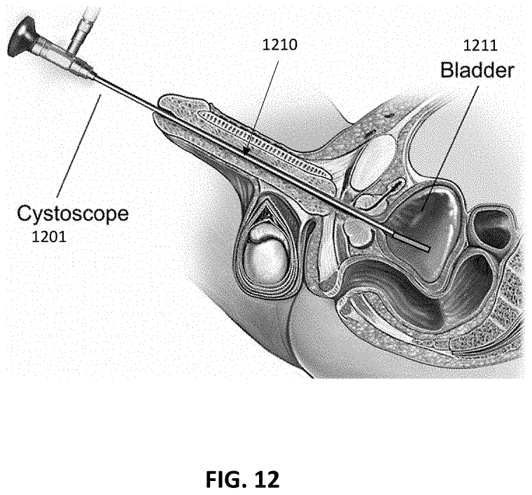

FIG. 12 illustrates the use of a biopsy device as described herein within the urethra 1210 and/or bladder 1211. In this example, the device may be used with another tool such as a cytoscope 1201.

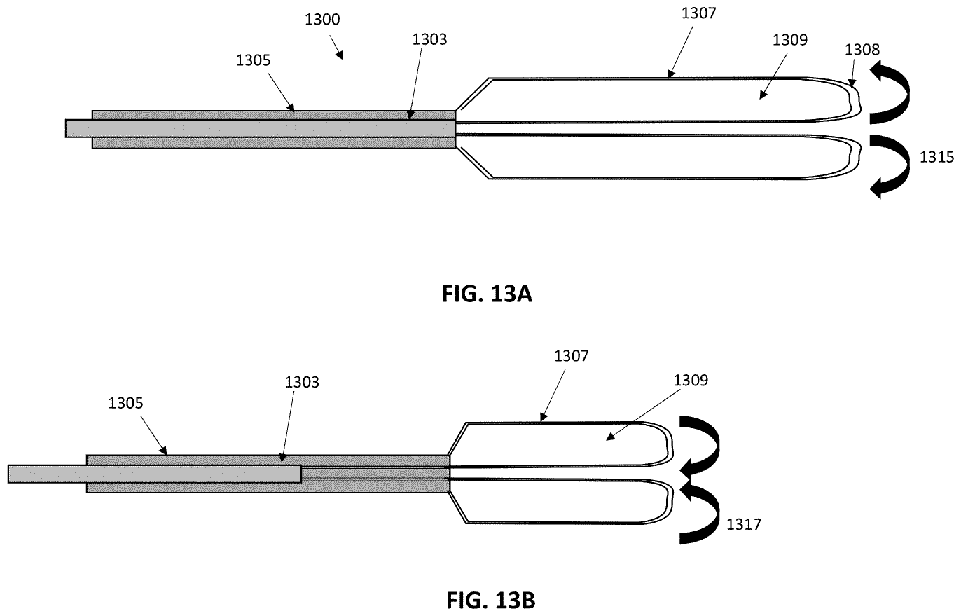

FIGS. 13A-13B illustrate one example of a biopsy apparatus as described herein with a line or additional biopsy collecting member 1309 included as part of the biopsy collector 1307. For example, as shown in FIG. 13A, the device 1300 may include an inner member 1303 and an outer member 1305 that are concentrically arranged. The inner member may be attached to a first end of the biopsy collector 1307 and the outer member may be attached to a second end of the biopsy collector, as shown. The biopsy collector in this example includes an outer (e.g., high friction, member 1308, which may be a woven, braided or knitted member as described above. Underneath this (or in some cases covering this) may be a material having higher affinity for collecting the biopsy material, such as a textile adapted to collect biopsy material. This may be configured as a sleeve or sock of fabric, and/or a liner. The liner can be everted with the rest of the biopsy collector and may be drawn back into the tube, as shown in FIG. 13B, by pulling the inner member proximally. Thus, the entire biopsy collector may be everted 1315 or rolled out by driving the inner member distally and/or pulled back into the outer member 1317 by pulling proximally. Any of the biopsy device variations described herein may include a liner, sleeve, etc.

In some variations, the liner or sleeve may be configured as a fabric liner. For example, a fabric liner may be a knitted material formed by knitting an 80 denier polyester, using a 22 needle machine. The liner outer diameter (OD) may be less than about 1 mm, constrained. An example of a braided liner may be formed of an 80 PET material having 48 ends (e.g., 48 filaments, which may be monofilaments or poly-filaments) that have the same dimensions as described above. For example, the ranges of dimensions may be, e.g., for knitted variations, between about 20-150 Denier, 12 needle to 40 needle (less needles for larger Denier yarns). For braided variations, between about 20-150 Denier, 24 to 72 ends (more ends less yarns). In some variations, including an ePTFE thin walled tube, the tube may have an outer diameter of about 2 mm, 0.001″ or thinner wall thickness. As mentioned above, the liner may be internal to the outer everting member (e.g., an inside liner) or external to the everting member (e.g., as a skin or covering). The inside liner may be useful for to trapping and/or containing biopsy tissue or cells that may move through the outer everting material. When the liner is outside of the biopsy collector element, it way function as a rough, highly porous cell capturing skin.

In general, the apparatuses described herein may be configured so that the biopsy collector, which may be woven or braided from a plurality of filaments, assumes a jammed configuration when un-inverted (including when within the outer cannula), in which the biopsy collector is prevented from collapsing beyond a minimum inner diameter in the un-deployed (and un-inverted) configuration. For example, a small-tube biopsy device may include: an inner cannula coaxially arranged within an outer cannula, so that the inner cannula may move axially in a distal and proximal direction relative to the outer cannula; a biopsy collector comprising a woven or braided mesh formed of a plurality of filaments, wherein the biopsy collector is porous and is connected at a first end to the inner cannula and at a second end to the outer cannula, further wherein the biopsy collector has a first, un-deployed, configured in which a majority of the biopsy collector is un-inverted (and in some variations may be at last partially retained within a lumen of the outer cannula), and a second, deployed, configuration in which the majority of the biopsy collector is inverted relative to the un-deployed configuration, and may be expanded beyond an outer diameter of the outer cannula; and a proximal handle coupled to the inner cannula and the outer cannula and configured to move the inner cannula axially relative to the outer cannula to transition biopsy collector between the first un-deployed configuration and the second deployed configuration, wherein the biopsy collector is configured so that the number and/or size (e.g., cross-sectional dimensions and/or shape) of the plurality of filaments maintains an inner diameter of the biopsy collector in the first, un-deployed configuration at greater than 40% (e.g., 50% or more, 55% or more, 60% or more, 70% or more, 75% or more, 80% or more, 85% or more, 90% or more, etc.) of an inner diameter of the outer catheter. Alternatively or additionally, the biopsy collector may be configured so that the number and/or size (e.g., cross-sectional dimensions and/or shape) of the plurality of filaments (e.g., wires) cause the biopsy collector to jam at a minimum diameter that is greater than or approximately the same (e.g., 80% or more, 85% or more, -90% or more 95% or more, 100% or more, etc.) of the inner diameter of the inner cannula when un-inverted.

Thus, any of these apparatuses described herein may include a biopsy collector that is porous and/or radio-opaque and/or configured to have a jammed configuration in the un-deployed configuration, when not-inverted, that maintains the radial patency of an inner lumen formed by the braided or woven biopsy collector that is continuous with an inner lumen of the inner cannula.

Although the biopsy collector may be biased to jam to a minimum inner diameter in the un-inverted configuration by heat setting the braided or woven filaments forming the biopsy collector, in some variations it may be particularly beneficial to configure the biopsy collector so that jamming is based on the number and/or size (e.g., cross-sectional dimensions and/or shape) and/or the arrangement (e.g., braid angle) of the plurality of filaments. For example, in some variations, particularly those configured to work with an outer member having a diameter of between 1 mm and 15 mm (e.g., between about 1 mm and about 12.5 mm, between about 1 mm and about 10 mm, between about 1 mm and 8 mm, between about 1 mm and 6 mm, etc.), the filaments may be, e.g., about 0.00025″ and about 0.05″ wires (e.g., between about 0.0005 to about 0.01″, between about 0.0005″ to about 0.005″, etc.), and the plurality of filaments may include 24 or more strands (e.g., 36 or more, 48 or more, 60 or more, 72 or more, 84 or more, etc.). At these combinations of filament number, filament size, and/or braid angle, the biopsy collector maybe prevented from collapsing to an inner diameter that is narrower than the jammed diameter (e.g., approximately 90% or more the inner diameter of the inner cannula to which the biopsy collector is attached). Thus, the jamming may be mechanically achieved, rather than relying on shape setting, which may otherwise be more prone to failure.

This minimum jammed diameter (minimum inner diameter) may provide a reliable lumen or channel that is continuous with the lumen of the inner cannula, which may ensure that a device, such as a guidewire, camera, biopsy tool, etc. may be passed through the device.

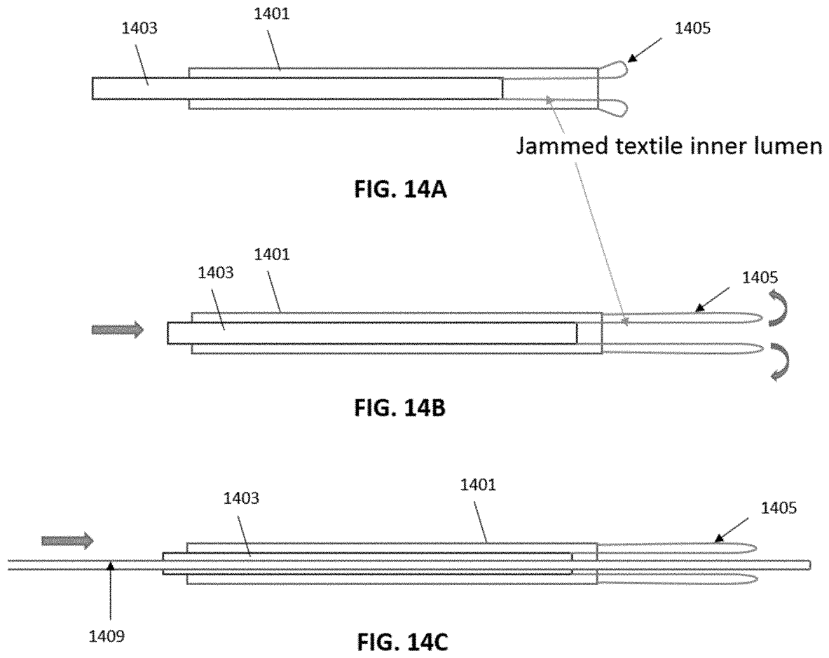

For example, FIGS. 14A-14C illustrate one example of a biopsy collection apparatus as described herein. In FIG. 14A, the apparatus (e.g., biopsy tool) includes an outer cannula (or other catheter) 1401 and an inner cannula (e.g., inner catheter) 1403. The inner cannula may also be referred to herein as an inner pusher, inner member or inner puller; the outer cannula may be referred to herein as an outer member or outer catheter. In FIG. 14A, a woven or braided biopsy collector 1405 is attached at either end to the distal end of the inner cannula and the distal end of the outer cannula. The biopsy collector may be referred to herein as an everting textile element. In FIG. 14A, the biopsy collector is shown in the un-deployed configuration, in which it is un-inverted, and is jammed so that it has a minimum inner diameter that is approximately the same as the inner diameter of the inner cannula, and greater than 50% of the inner diameter of the outer cannula (e.g., 50% or more, 60% or more 70% or more 75% or more, 80% or more, etc.).

As shown in FIG. 14B, the jammed configuration of the un-inverted region of the woven or braided biopsy collector 1405 is maintained even as the device is deployed by driving the inner cannula distally, as shown. In FIG. 14B, even as the biopsy collector is deployed form out of the outer cannula and extended outwards (in some variations expanding radially outwards, not shown in FIG. 14B), the inner lumen of the biopsy collector may be jammed to prevent it from assuming an inner diameter that is less than the minimum jammed diameter (e.g., approximately the same as the inner diameter of the inner cannula and/or 50% or more of the inner diameter of the outer cannula, etc.). This minimum jammed diameter may be due to the mechanical jamming of the plurality of filaments forming the biopsy collector as mentioned above. In addition, the minimum jammed diameter may provide an optimal lumen for passing one or more additional tools (e.g., biopsy tool, snare, camera, video, RF tool, lavage, applying drug(s), contrast, etc.) through the lumen of the inner cannula and biopsy collector. This is illustrated, e.g., in FIG. 14C, showing an adjunct tool 1409 passing through the lumen formed by the inner cannula 1403 and the un-inverted portion of the biopsy collector 1405.

The biopsy collector may be braided or woven at any appropriate filament (e.g., braid) angle. The filament angle may help set the minimum jamming diameter. In general, these biopsy collectors may be deployed without requiring or implicating hydraulic pressure; they are not typically hermetically sealed, but instead are porous and/or permeable. The outward radial force of the biopsy collector may be controlled by the material stiffness of the filament material, e.g., stainless steel, Nitinol, etc. As mentioned, the material dimensions (e.g., the diameter and/or shape, such as round cross-section, square cross-section, rectangular cross-section, etc.), and/or the number of wires. In particular, the number of wires may be determinative.

The biopsy collector typically forms an everting tube that everts (inverts) over onto itself. The everted tube column force may be defined, at least in part, by the outward radial force and textile filament (e.g., braid) angle. For example, the higher braid angle may create an outer tube jammed column having a higher column force with a relatively low bending stiffness.

Any of the apparatuses described herein may include a biopsy collector that does not expand substantially (e.g., similar to that shown in FIG. 14A-14C). For example the expanded configuration of the biopsy collector may be configured to be approximately the same outer diameter as the outer catheter; in some variations the expanded configuration of the biopsy collector has an outer dimeter that is between about 1.1× and 2× the outer diameter of the outer catheter, between about 1.1× and 1.5× the outer diameter of the outer catheter, etc.

The methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

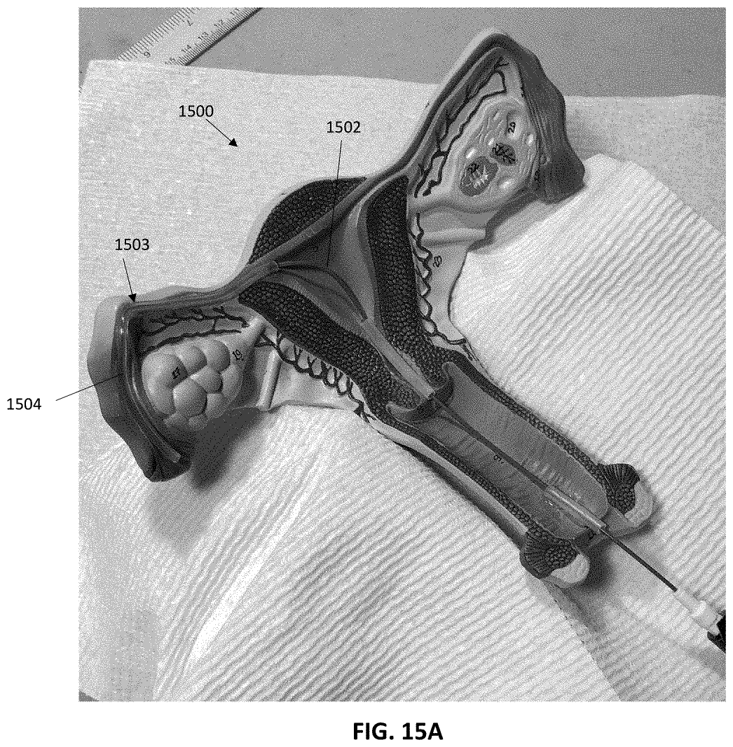

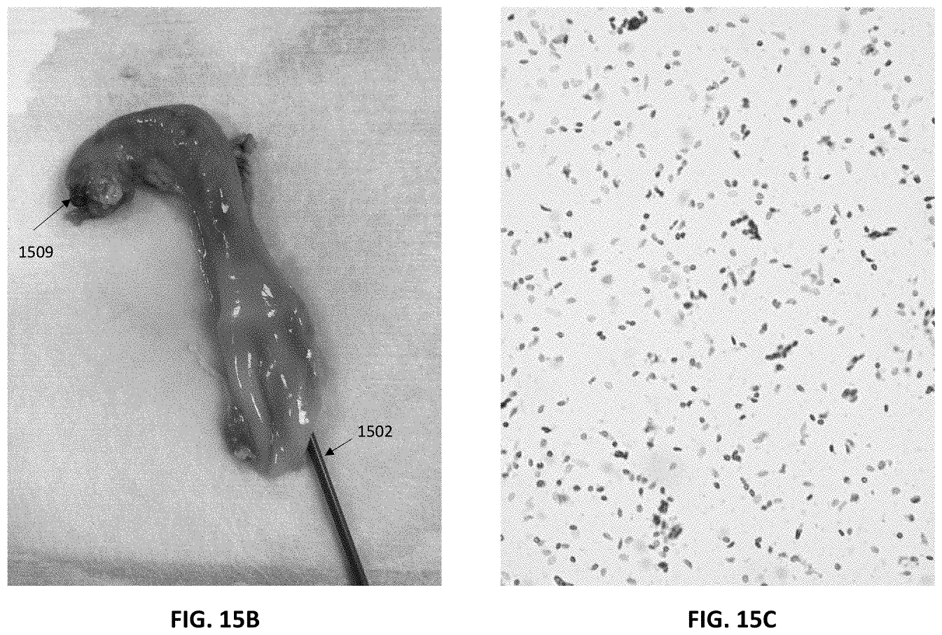

FIG. 15A shows a scale model of a female reproductive tract 1500 in which a prototype device is deployed into one of the fallopian tubes 1503. In this example, the prototype device includes an outer catheter 1502, biopsy collector formed of woven or braided filaments as described above 1504, and an inner pusher (not visible). In practice, this device may be deployed using fluoroscopy and/or ultrasound to visualize the apparatus being deployed. As mentioned above, the apparatus maybe configured to be visible under ultrasound (e.g., the biopsy collector) and/or fluoroscopy.

FIG. 15B illustrates one example of a prototype apparatus such as those illustrated above, being used to biopsy a fetal bovine reproductive tract. In FIG. 15B the apparatus has been deployed within the fallopian tube by first extending the apparatus with the biopsy collector retracted within the outer catheter; the device may be extended directly or over a guidewire/guide catheter (not shown). Once the distal end of the outer catheter is near the start of the sampling region (e.g., the opening of the fallopian tube) the inner member may be pushed distally to evert the biopsy collector out of the apparatus and into the fallopian tube as described above. The compliant outer surface of the biopsy collector may conform to the walls of the fallopian tube, allowing it to sample material. In FIG. 15B, the distal end of the biopsy collector has been extended to the tip of the distal fimbriae 1509. The device may then be retracted into the outer catheter 1502 and withdrawn. After being withdrawn, collected cells may be extracted from the biopsy collector, for example by extending the biopsy collector within a collection media (e.g., liquid media) and or applying to a plate/slide, for histological examination. FIG. 15C shows an example of bovine cells collected (and stained) at 100x magnification, from a preparation such as that illustrated in FIG. 15B.

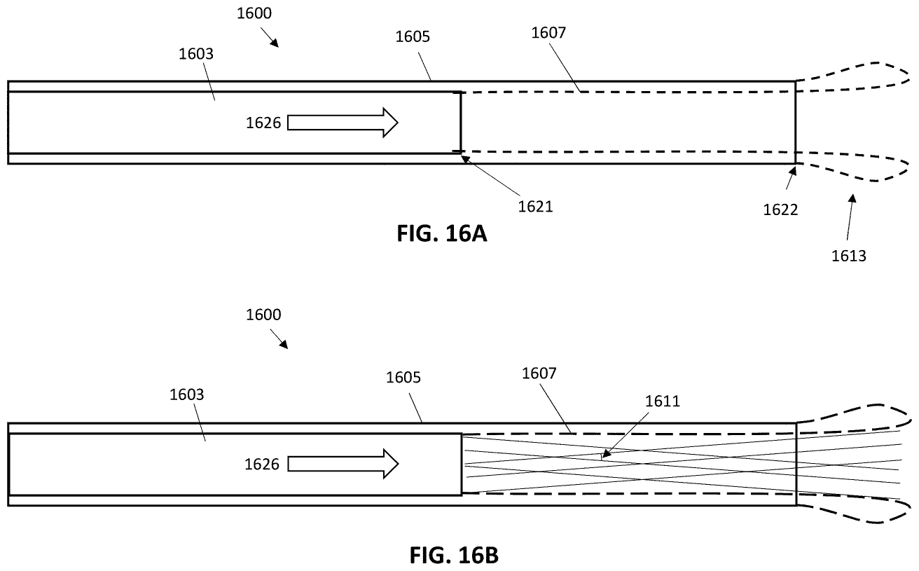

FIGS. 16A-16B illustrate an additional examples of a biopsy device having an inner member 1603 that is coaxially arranged within an outer member 165 (e.g., or outer cannula), so that the inner member may move axially in a distal and proximal direction relative to the outer member. In FIG. 16A the biopsy device is shown partially deployed, with the biopsy collector 1607 (comprising a tube of knitted textile) driven 1628 distally out of the end of the outer member to that the tube of knitted material extends distally 1613 relative to the distal end opening of the outer member. In FIG. 16A the biopsy collector is a knitted material that is configured to collect biopsy tissue (e.g., within pores formed by the gaps between the knitted strands forming the tube). The biopsy collector is connected at a first end 1621 to the inner member and at a second end 1622 to the outer member. In the undeployed configuration, all or a majority of the biopsy collector 1607 may be retained within the lumen of the outer member 1605, which may protect it. Any of these device, including the one shown in FIG. 16A-16B, may include a proximal handle (not shown) that is coupled to the inner member and the outer member and configured to move the inner member distally 1626 relative to the outer member so that the biopsy collector extends distally as an inverted tube 1613 out of a distal end of outer member and everts over itself. Handle may include a stop limiting the distal movement of the inner member.

In any of the devices described herein, the biopsy collector may include a braided textile having a braid angle in the un-deployed configuration of 25 degrees or greater, and an outer diameter 85% or greater of the inner diameter of the outer member (e.g., 90% or greater, between about 0.9 and 0.999 times the inner diameter of the outer member) in the un-deployed configuration. This is illustrated in FIG. 16B. In FIG. 16B, the device 1600 include a tube of braided material having a braid angle 1611 that is 25 degrees or less (e.g., less than 25 degrees, less than 24 degrees, less than 23 degrees, less than 22 degrees, less than 21 degrees, etc.). The low braid angel in the un-deployed configuration within the lumen of the outer member (as shown) may provide a good column force on the tube of woven material, particularly where there are between about 20-50 strands of, e.g., about 0.002 inch wire (e.g., between about 24-48 strands, etc.), having a diameter of between about 0.55 mm and 0.70 mm inner diameter (e.g., between about 0.60-0.65 mm, etc.). This stable cylinder (tube) of woven material may be pushable. In FIG. 16B the tube is shown to fit snugly within the inner lumen of the outer member 1605, preventing kinking of the tube of braided material. For example in FIG. 16B the outer diameter of the un-inverted (un-deployed) tube of braided material within the outer member 1605 is about 90% (e.g., between 90%-99% of the inner diameter of the outer member. The braid angle 1611 in FIG. 16B refers to the distal-facing angle of the strands, as shown.

In use, this apparatus may be used to take a biopsy from within a body lumen; the user (e.g., doctor) may grasp the handle and/or secure the outer member while advancing the inner member distally to extend the tube of braided material distally out of the end of the outer member and into the lumen (the outer member may remain outside of the lumen or may stay fixed relative to the lumen), as described above.

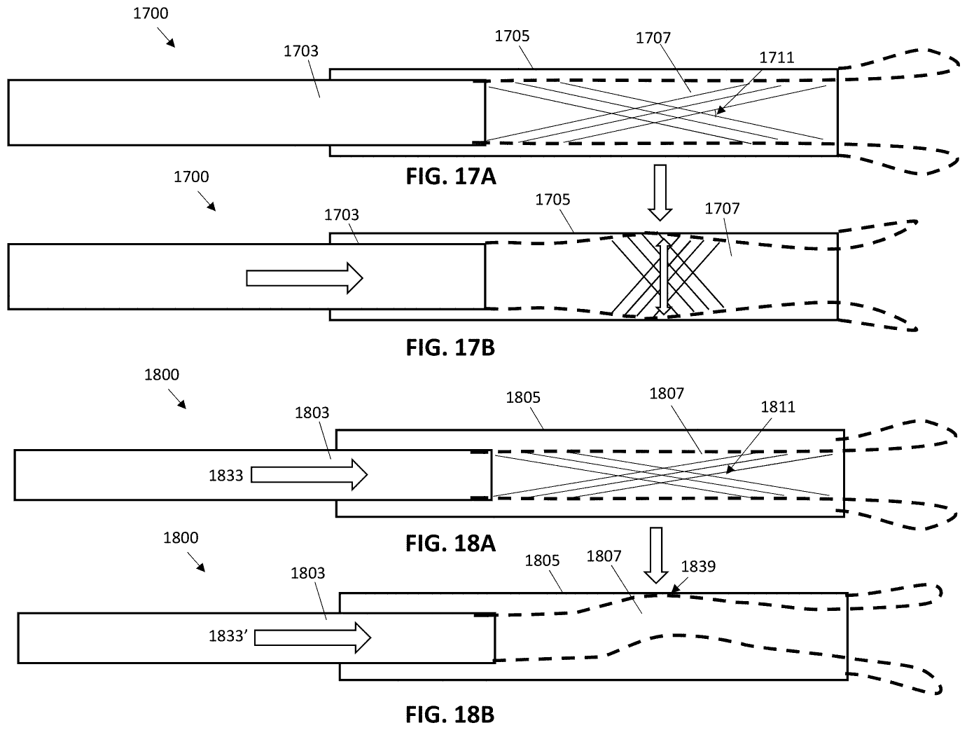

Biopsy devices 1700 in which the braid angle of the tube of braided textile has a small braid angle may prevent locking up (e.g., “cleating”) of the tube of material within the lumen of the outer member during operation of the device. This is illustrated in FIG. 17A-17B. In FIG. 17A the braid angle 1711 of the braided textile forming the biopsy collector 1707 is greater than 25 degrees in the un-deployed configuration within the outer member 1705. When the inner member 1703 is pushed distally, as shown in FIG. 17B, the portion of the biopsy collector 1707 within the outer member 1705 has a relatively low column strength, and may bugle 1734, as shown, preventing the biopsy collector from extending distally out of the outer member to collect biopsy material. In FIGS. 17A-17B the biopsy collector is formed of a tube of braided textile that is unstable when pushed (e.g., under compression).

FIGS. 18A-18B illustrate another example of a potential failure mode for an apparatus 1800 as described herein. In FIG. 18A, the device 1800 includes an inner member (pusher 1803) and an outer member 1805, the ends of which are coupled to the ends of a tube of braided textile forming the biopsy collector 1807. As in FIGS. 16B and 17A-17B, only some of the strands of the braided textile are shown. In FIG. 18A, the braided textile has a braid angle 1811 of 25 degrees of less (e.g., <25 degrees, <24 degrees, <23 degrees, etc.) which otherwise provides a relatively good column strength when the pushing the inner member distally to evert the biopsy collector out of the distal end. However, as shown in FIGS. 18A-18B, the biopsy collector in the un-deployed configuration (within the lumen of the outer member)is loosely held within the lumen of the outer member. In this example, the outer diameter of the tube of braided textile in the un-deployed configuration is less than 85% (e.g.,. less than 90%, less than 80%, less than 75%, less than 70%, etc.) of the inner diameter of the outer tube 1805 when the braided textile is under compression (e.g., by pushing it distally) 1833. As shown in FIG. 18B, this may allow the tube of braided textile within the outer member to buckle 1839. Buckling may jam or prevent the device from extending the biopsy collector distally out of the distal end of the outer member. Alternatively of additionally, in some variations, buckling due to either the higher braid angle and/or a looser fit within the outer catheter may be addressed and ameliorated by increasing the stiffness of the strands (e.g. wires, including increasing the diameter, altering the material, etc.), increasing lubricousness of the inner walls of the outer member, etc.

Any of the apparatuses described herein may alternatively or additionally control the braid angle of the inverted biopsy collector, e.g., in a knitted, braided or woven device. For example, a braided textile forming the biopsy collector may be configured to have a high braid angle of, e.g., 125 degrees or more in the everted configuration that is extending distally from the outer member. This braid angle may be a post-annealed (e.g., shape-set) angle.

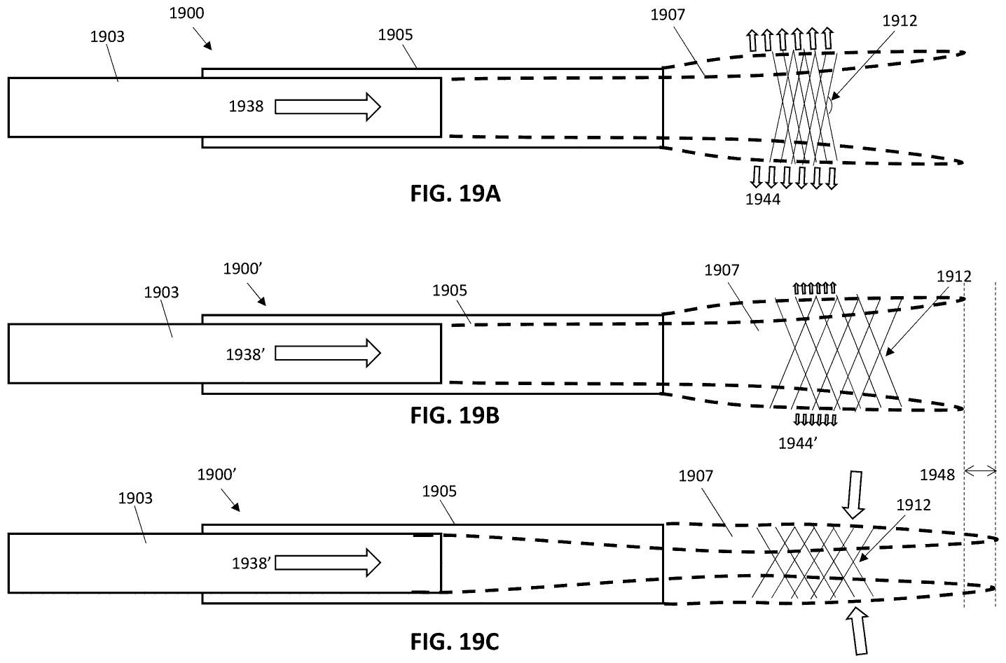

For example, FIG. 19A shows one example of a device 1900 having an inner member (pusher 1903) coupled at a distal end to a biopsy collector 1907 shown as a tube of braided textile in this example. The opposite end of the biopsy collector 1907 is coupled to the distal end region of an outer member 1905. In FIG. 19A, the everted braid angle 1912 is about 125 degrees or greater. In this configuration the larger braid angle on the everted (e.g., deployed) biopsy collector provides high degree of radial force and more stable rolling tube, which resists stretching. This configuration may also provide the un-everted (e.g., un-deployed) portion of the tube of braided textile with the desired braid angle (e.g., of 25 degrees or less) as mentioned above. The radial force (arrows 1944) on the braided region distally extending from the distal end of the outer member when the inner member is driven distally 1938 may help drive the biopsy collector against the tissue and may further assist in collecting tissue (e.g., within the gaps, also referred to as pores, formed between the strands making the braid).

FIGS. 19B and 19C illustrate an example of a device that does not include a large braid angle (e.g., having an everted braid angle 1912′ of less than) may have a low radial force and may not resist stretching as well as the device in FIG. 19A. As shown in comparison between FIG. 19B and 19C, when the inner member 1903 of the device 1900′ is advanced distally 1938′ the biopsy collector 1907 may stretch (as shown by lines 1948) and may have a lower radial force 1944′ and therefore a less stable rolling biopsy collector 1907.

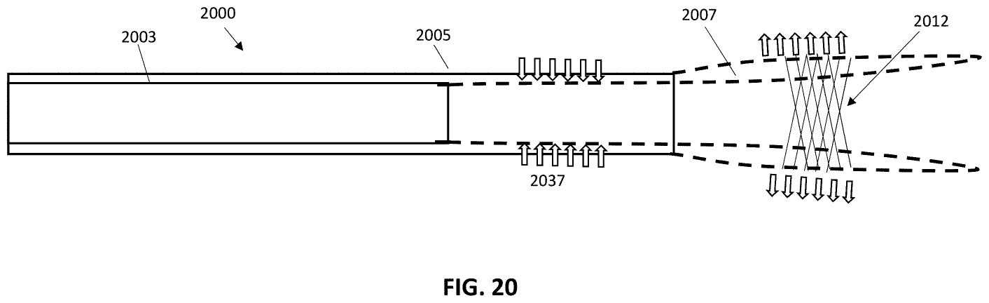

FIG. 20 illustrates another example of a device having a braided tube forming the biopsy collector with a high everted braid angle (e.g., >125 degrees) which may resist stretching, provide a high radial force. In some cases, when the everted braid is greater than about 125 degrees, when it is inverted back into the outer member 2005 (e.g., outer catheter) by pulling on the inner member 2003, the inverted braid may create a collapsing force 2037, which may squeeze down into a jammed column and resists expansion when compressed (e.g., when deployed).

In any of the apparatuses (e.g., devices, systems, etc.) described herein, the apparatus may have a biopsy collector that is between about 5 mm and 40 mm long (e.g., between about 5 and 30 mm long, between about 10-30 mm long, etc.). In addition, the apparatus may be configured so that only about half of the biopsy collector is extended from the distal end of the outer member (e.g., catheter), providing an effective biopsy collector length of between about 2.5-20 mm long (e.g., between about 5-20 mm long, between about 7-15 mm long, etc.). Other lengths may be used.

For example, some variations of these apparatuses may be configured as fallopian biopsy devices. Such devices may have, for example, an outer diameter of the outer member (of between about 1 mm and about 1.5 mm (e.g., between about 1.1-1.3 mm, between about 1.2-1.4 mm, about 1.1 mm, about 1.2 mm, about 1.3 mm, about 1.4 mm, about 1.5 mm, etc.). The outer diameter of the inner member may be, for example, between about 0.5 mm and about 0.9 mm (e.g., between about 0.5 and about 0.75 mm, between about 0.6 and about 0.8 mm, etc.). The inner and outer members may be any appropriate length and flexibly to allow the biopsy device to be delivered to the desired body lumen. For example, the length of the outer member may be between 10 cm and 1 meter. The inner member may be longer than the outer member. The outer member may be a cannula. The inner member may be a rod, wire, cannula, hypotube, etc. The handle may limit the extension (e.g., fully extended length) of the biopsy collector to the desired length (e.g., 5 cm or less, 7.5 cm, 10 cm, 12 cm or less, etc.)

As mentioned above, the biopsy collector may be formed as a textile of knitted, woven or braided material. The material forming the textile may be a polymeric material and/or a metallic material (e.g. Nitinol). In some examples, the biopsy collector is a braided material formed by between 20-60 strands of braided wire, such as 0.002 inch wire (e.g., between about 0.0015 inch wire and 0.0025 inch diameter wire). In some variations the biopsy collector has an outer diameter of 0.025 inches (approximately 0.63 mm in the inner diameter). For example, the apparatus may include a braided tube forming a biopsy collector having between about 24 and 48 strands. Lower strand numbers may provide a higher porosity (e.g., larger sized pores or openings).

Any of the apparatuses described herein may be adapted to biopsy other regions of the body, including, for example, the uterus (e.g., endometrial biopsy), urethra, nose, sinus cavity, GI tract (e.g., colon, rectum, small intestine, etc.), esophagus, ear, etc. Thus, any of the devices described herein may be scaled to biopsy from these regions. Scaling may be linear in size; in variations in which the biopsy collector is formed of multiple knitted, woven or braided strands, the number of strands may be increased or decreased accordingly (as the device is scaled). One exemplary device may include a biopsy collector having between 65-80 (e.g., about 72) strands of 0.004″ Nitinol, having an everted outer diameter of about 15 mm, and an everted braid angle of greater than 125 degrees. This device may be used for uterine biopsy (e.g., endometrial biopsy). Another example may have a biopsy collector having between 135-155 (e.g., about 144) strands of 0.004″ Nitinol, having an everted outer diameter of about 22 mm, and an everted braid angle of greater than 125 degrees. The inner member may be a 5F (e.g., 0.065″ OD inner catheter or rod) and the outer member may be a catheter having a 7F (e.g., 0.091″ OD).

In general the strands forming the biopsy collector may be any shape (cross-sectional shape), including round, oval, square, triangular, etc. as mentioned. These wires may be radiopaque to aid in visualization (e.g., under fluoroscopy, ultrasound, etc.) or may include other markers.

Any of these methods may be used in conjunction with a dye, contrast, or the like. For example, a dye may be delivered through the inner member. As mentioned above, any of these device may also or alternatively be used to help open a collapsed tube or lumen.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of collecting biopsy material from within a fallopian tube, the method comprising:
advancing a biopsy device distally towards an opening into the fallopian tube;
extending a tube of braided textile distally into the fallopian tube by moving an inner member of the biopsy device distally within an outer member of the biopsy device, wherein a first end region of the braided textile is attached to a distal region of the inner member and a second end region of the braided textile is attached to a distal region of the outer member, so that the tube of braided textile extends distally from a distal end of the outer member and everts over itself as it extends into the fallopian tube;
capturing cellular material from the fallopian tube on pores within the braided textile; and
withdrawing the inner member of the biopsy device proximally so that the tube of braided textile is inverted and drawn proximally back into the lumen of the outer member.

2. The method of claim 1, wherein capturing the cellular material comprises moving the tube of braided textile against an inner wall of the fallopian tube to capture the cellular material.

3. The method of claim 1, wherein advancing the biopsy device comprises passing the biopsy device over a guidewire positioned in the body lumen.

4. The method of claim 1, wherein advancing comprises advancing the biopsy device with the tube of braided textile completely within the outer member.

5. The method of claim 1, wherein capturing cellular material from the body lumen comprises applying compressive force on the braided textile to form a stable column of braided material in an un-inverted configuration within the outer member.

6. The method of claim 1, wherein the tube of braided textile forms a distally-extending torus as it extends out of the distal end of the outer member.

7. The method of claim 1, wherein capturing comprises holding the outer member relative fixed and advancing the inner member distally to extend the tube of braided textile distally into the fallopian tube.

8. The method of claim 1, further comprising moving the tube of braided textile by: rotating the tube of braided textile relative to the fallopian tube, pulling the tube of braided textile proximally relative to the fallopian tube, and/or pushing the braided textile distally relative to the fallopian tube.

9. A fallopian tube biopsy device, the device comprising:
an inner member coaxially arranged within an outer member, so that the inner member may move axially in a distal and proximal direction relative to the outer member;
a biopsy collector comprising a knitted, braided or woven textile configured to collect biopsy tissue, wherein the biopsy collector is connected at a first end to the inner member and at a second end to the outer member and wherein a majority of the biopsy collector is retained within a lumen of the outer member in an un-deployed configuration; and
a proximal handle coupled to the inner member and the outer member and configured to move the inner member distally relative to the outer member so that the biopsy collector extends distally out of a distal end of outer member and everts over itself.

10. The device of claim 9, further comprising a stop on the proximal handle limiting the distal movement of the inner member.

11. The device of claim 9, wherein the knitted, braided or woven textile comprises a porosity that is configured to enhance collection of biopsy tissue.

12. The device of claim 9, wherein the pores of the knitted, braided or woven textile are between about 0.002″ and 0.05″.

13. The device of claim 9, wherein the biopsy collector is configured to prevent the knitted or woven textile from rolling against a distal end of the outer member.

14. The device of claim 9, wherein the biopsy collector is connected at the first end to the distal end of the inner member.

15. The device of claim 9, wherein the biopsy collector is connected at the second end to a distal end of the inner member.

16. The device of claim 9, wherein the proximal handle is configured to move the inner member axially relative to the outer member to expand the biopsy collector so that the outer diameter of the biopsy collector is enlarged to contact the inner walls of a body lumen.

17. The device of claim 9, wherein the knitted, braided or woven textile is attached to an inner surface of the outer member.

18. The device of claim 9, further comprising a guidewire lumen thought the inner member and the biopsy collector.

19. The device of claim 9, wherein the proximal handle comprises a control configured to move the inner member axially distally and proximally relative to the outer member while holding the outer member in a fixed position.

20. The device of claim 9, wherein the biopsy collector compromises a braided textile having a braid angle in the un-deployed configuration of 25 degrees or greater, and an outer diameter that is between 0.9 and 0.99 times the inner diameter of the outer member in the un-deployed configuration.

21. The device of claim 9, wherein the biopsy collector comprises a braided textile having a braid angle in a deployed and inverted configuration that is 125 degrees or greater.

22. A small-tube biopsy device comprising:
an inner member coaxially arranged within an outer member, so that the inner member may move axially in a distal and proximal direction relative to the outer member;
a biopsy collector comprising a braided textile having a plurality of pores that are configured to collect biopsy tissue, wherein the biopsy collector is connected at a first end to the inner member and at a second end to the outer member and wherein a majority of the biopsy collector is retained within a lumen of the outer member in an un-deployed configuration; and
a proximal handle coupled to the inner member and to the outer member and configured to move the inner member distally relative to the outer member so that the biopsy collector extends distally out of a distal end of outer member and everts over itself to assume a deployed configuration that extends distally away from the distal end of the outer member, wherein the braided textile is configured to have a braid angle of 125 degrees or more in the deployed configuration.
